(19) United States
(12) Patent Application Publication
(10) Pub. No.: US 2008/0200366 A1
(43) Pub. Date: Aug. 21, 2008

(54) Trimeric Peptides for Use in Treating Obesity

(75) Inventor: Kilian Waldemar Conde Frieboes, Malov (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/911,428

(22) PCT Filed: Apr. 7, 2006
(86) PCT No.: PCT/EP06/61465

§ 371 Date: Nov. 29, 2007

(30) Foreign Application Priority Data
Apr. 14, 2005 (DK) PA 2005 00540

(51) Int. Cl. A61K 38/02; C07K 2/00; A61P 3/04; A61P 3/00

(52) U.S. Cl. 514/2; 530/300

(57) ABSTRACT

The present invention relates to novel peptide compounds which are effective in modulating one or more melanocortin receptor types, to the use of the compounds in therapy, to methods of treatment comprising administration of the compounds to patients in need thereof, and to the use of the compounds in the manufacture of medicaments. The compounds of the invention are of particular interest in relation to the treatment of obesity as well as a variety of diseases or conditions associated with obesity.

### FIELD OF THE INVENTION

The present invention relates to peptides which are conjugated to a central trimerisation moiety.

### BACKGROUND OF THE INVENTION

Obesity is a well known risk factor for the development of many very common diseases such as atherosclerosis, hypertension, type 2 diabetes (non-insulin dependent diabetes mellitus (NIDDM)), dyslipidaemia, coronary heart disease, and osteoarthritis and various malignancies. It also causes considerable problems through reduced motility and decreased quality of life. The incidence of obesity and thereby also these diseases is increasing throughout the entire industrialised world. Only a few pharmacological treatments are available to date, namely Sibutramine (Abbot; acting via serotonergic and noradrenaline mechanisms) and Orlistat (Roche Pharm; reducing fat uptake from the gut,). However, due to the important effect of obesity as a risk factor in serious (and even fatal) and common diseases, there is still a need for pharmaceutical compounds useful in the treatment of obesity.

The term obesity implies an excess of adipose tissue. In this context, obesity is best viewed as any degree of excess adiposity that imparts a health risk. The distinction between normal and obese individuals can only be approximated, but the health risk imparted by obesity is probably a continuum with increasing adiposity. However, in the context of the present invention, individuals with a Body Mass Index (BMI=body weight in kilograms divided by the square of the height in meters) above 25 are to be regarded as obese.

Even mild obesity increases the risk for premature death, diabetes, hypertension, atherosclerosis, gallbladder disease and certain types of cancer. In the industrialized western world the prevalence of obesity has increased significantly in the past few decades. Because of the high prevalence of obesity and its health consequences, its treatment should be a high public health priority.

When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity.

Pro-opiomelanocortin (POMC) is the precursor for β-endorphin and melanocortin peptides, including melanocyte stimulating hormone (α-MSH) and adrenocorticotropin (ACTH). POMC is expressed in several peripheral and central tissues including melanocytes, the pituitary, and neurons of the hypothalamus. The POMC precursor is processed differently in different tissues, resulting in the expression of different melanocortin peptides depending on the site of expression. In the anterior lobe of the pituitary, mainly ACTH is produced whereas in the intermediate lobe and the hypothalamic neurons the major peptides are α-MSH, β-MSH, desacetyl-α-MSH and β-endorphin. Several of the melanocortin peptides, including ACTH and α-MSH, have been demonstrated to have appetite-suppressing activity when administered to rats by intracerebroventricular injection [Vergoni et al, European Journal of Pharmacology 179, 347-355 (1990)]. An appetite-suppressing effect is also obtained with the artificial cyclic α-MSH analogue, MT-II.

A family of five melanocortin receptor subtypes has been identified (melanocortin receptor 1-5, also called MC1, MC2, MC3, MC4 and MC5). The MC1, MC2 and MC5 are mainly expressed in peripheral tissues whereas MC3 and MC4 are mainly centrally expressed, however MC3 are also expressed in several peripheral tissues. MC3 receptors have besides being involved in energy homeostasis also been suggested to be involved in several inflammatory diseases. An MC3 agonist could have a positive effect on these diseases, e.g. gouty arthritis. MC5 are mainly peripheral expressed and has been suggested to be involved in exocrine secretion and in inflammation. MC4 is shown to be involved in the regulation of body weight and feeding behaviour as MC4 knock out mice develop obesity (Huzar et al, Cell 88, 131-141 (1997)). Furthermore studies of either ectopic centrally expression of agouti (MC1, MC3 and MC4 antagonist) or over-expression of an endogenously occurring MC3 and MC4 antagonist (agouti gene related peptide, AGRP) in the brain demonstrated that the over-expression of these two antagonists lead to the development of obesity (Kleibig et al, PNAS 92, 4728-4732 (1995)). Furthermore, icv injection of a C-terminal fragment of AGRP increases feeding and antagonises the inhibitory effect of α-MSH on food intake.

In humans several cases of families with obesity presumably due to frame shift mutations in MC4 have been described (e.g. Yeo et al, Nature Genetics 20, 111-112 (1998), Vaisse et al, Nature Genetics 20, 113-114).

In conclusion, a MC4 agonist could serve as an anorectic drug, and be useful in the treatment of obesity or obesity related diseases as well as in the treatment of other diseases, disorders or conditions, which are improved by activation of MC4.

MC4 antagonists may be useful for treatment of cachaxia, anorexia, and for treatment of waisting in frail elderly patients. Furthermore, MC4 antagonists may be used for treatment of chronic pain, neuropathy and neurogenic inflammation.

The present inventors have surprisingly found that specific peptides when applied to a central trimeric molecule has improved properties such as a high modulating effect on one or more melanocortin receptors, i.e. the MC1, MC2, MC3, MC4 or MC5 receptors.

### SUMMARY OF THE INVENTION

In one embodiment of the present invention, the compound is a ligand of a melanocortin receptor.

In one embodiment of the present invention, the compound is a ligand of the MC-4 receptor.

In one embodiment of the present invention, the compound is an agonist of the MC-4 receptor.

In one embodiment of the present invention, the compound is a selective agonist of the MC4 receptor. In this context, selectivity is to be understood in relation to the activity of the compound with respect to MC1, MC3 and/or MC5. If a compound is a significantly more potent MC4 agonist than it is a potent MC1, MC3 and/or MC5 agonist, it is deemed to be a selective MC4 agonist. The potencies of a compound with respect to MC1 and MC4 are determined in receptor binding assays as described in assay IV (MC 1) and assay V (MC4). If a compound is more than 10 times, such as more than 50 times, such as more than 100 times more potent with respect to MC4 than with respect to MC1, it is deemed to be a selective MC4 agonist with respect to MC1. The potencies of a compound with respect to MC3, MC4 and MC5 are determined in functional assays as described in assay II (MC 3 and MC5) and assay III (MC4). If a compound is more than 10 times, such as more than 50 times, such as more than 100 times more potent with respect to MC4 than with respect to MC3, it is deemed to be a selective MC4 agonist with respect to MC3. If a compound is more than 10 times, such as more than 50 times, such as more than 100 times more potent with respect to MC4 than with respect to MC5, it is deemed to be a selective MC4 agonist with respect to MC5. In a particular embodiment, the compound of the present invention is a selective MC4 agonist with respect to MC1, with respect to MC3, with respect to MC5, with respect to MC1 and MC3, with respect to MC1 and MC5, with respect to MC3 and MC5 or with respect to MC1, MC3 and MC5.

In one embodiment, the compound of the present invention is a selective MC4 agonists and a MC3 antagonist. in this context, a compound is deemed to be a selective MC4 agonist and a MC3 antagonist if it is a selective MC4 agonist with respect to MC1 and MC5 as discussed above, and it antagonizes MC 3 measured as described in assay II. A compound with an IC₅₀ value less than 100 nM, such as less than 10 nM, such as less than 5 nM, such as less than 1 nM is deem to be a MC3 antagonist.

In one embodiment, the compound of the present invention is both a selective MC3 agonist and a selective MC4 agonist. In this context, a compound is deemed to be a selective MC3 and MC4 agonist if it is significantly more potent MC3 and MC4 agonist than it is a potent MC1 and MC5 agonist. The selectivity of a compound with respect to MC1 and MC3 are determined by comparing the potency determined for MC1 as described in assay IV with the potency for MC3 determined as described in assay II. If a compound is more than 10 times, such as more than 50 times, such as more than 100 times more potent with respect to MC3 than with respect to MC1 it is deemed to be a selective MC3 agonist with respect to MC1. The selectivity of compound with respect to MC3 and MC5 are determined by comparing the potency determined as described in assay II. If a compound is more than 10 times, such as more the 50 times, such as more than 100 times more potent with respect to MC3 than with respect to MC5 it is de to a selective MC3 agonist with respect to MC5 receptor. The MC4 selectivity of a compound with respect to MC3 and MC5 is determined as discussed above.

Compounds of the present invention modulate melanocortin receptors, and they are therefore believed to be particular suited for the treatment of diseases or states which benefit from a modulation of the melanocortin receptor activity. In particular, compounds of the present invention are believed to be suited for the treatment of diseases or states which benefit from an activation of the MC-4 receptor.

In one embodiment, the present invention provides a method of delaying the progression from impaired glucose tolerance (IGT) to type 2 diabetes, the method comprising administering to a patient in need thereof an effective amount of a compound of the present invention, optionally in combination with one or more additional therapeutically active compounds.

In one embodiment, the present invention provides a method of delaying the progression from type 2 diabetes to insulin requiring diabetes, the method comprising administering to a patient in need thereof an effective amount of a compound of the present invention, optionally in combination with one or more additional therapeutically active compounds.

In one embodiment, the invention relates to a method of treating obesity or preventing overweight, the method comprising administering to a patient in need thereof an effective amount of a compound of the present invention, optionally in combination with one or more additional therapeutically active compounds.

In one embodiment, the present invention provides a method of regulating appetite, the method comprising administering to a patient in need thereof an effective amount of a compound of the present invention, optionally in combination with one or more additional therapeutically active compounds.

In one embodiment, the present invention relates to a method of inducing satiety, the method comprising administering to a patient in need thereof an effective amount of a compound of the present invention, optionally in combination with one or more additional therapeutically active compounds.

In one embodiment, the invention relates to a method of preventing weight regain after successfully having lost weight, the method comprising administering to a patient in need thereof an effective amount of a compound of the present invention, optionally in combination with one or more additional therapeutically active compounds.

In one embodiment, the invention relates to a method of increasing energy expenditure, the method comprising administering to a patient in need thereof an effective amount of a compound of the present invention, optionally in combination with one or more additional therapeutically active compounds.

In one embodiment, the present invention provides a method of treating a disease or state related to overweight or obesity, the method comprising administering to a patient in need thereof an effective amount of a compound of the present invention, optionally in combination with one or more additional therapeutically active compounds.

In one embodiment, the invention relates to a method of treating bulimia, the method comprising administering to a patient in need thereof an effective amount of a compound of the present invention, optionally in combination with one or more additional therapeutically active compounds.

In one embodiment, the invention relates to a method of treating binge-eating, the method comprising administering to a patient in need thereof an effective amount of a compound of the invention, optionally in combination with one or more additional therapeutically active compounds.

In one embodiment, the invention relates to a method of treating a disease or state selected from atherosclerosis, hypertension, diabetes, type 2 diabetes, impaired glucose tolerance (IGT), dyslipidemia, coronary heart disease, gallbladder disease, gall stone, osteoarthritis, cancers (in particular endometrial, breast, colon and prostate cancer), sexual dysfunction and risk of premature death, the method comprising administering to a patient in need thereof an effective amount of a compound of the present invention, optionally in combination with one or more additional therapeutically active compounds.

In particular, compounds of the present invention may be suited for the treatment of diseases in obese or overweight patients. Accordingly, the present invention also provides a method of treating, in an obese patient, a disease or state selected from type 2 diabetes, impaired glucose tolerance (IGT), dyslipidemia, coronary heart disease, gallbladder disease, gall stone, osteoarthritis, cancer (in particular endometrial, breast, colon and prostate cancer), sexual dysfunction and risk of premature death, the method comprising administering to a patient in need thereof an effective amount of a compound of the present invention, optionally in combination with one or more additional therapeutically active compounds.

In addition, MC4 receptor agonist could have a positive effect on insulin sensitivity, drug abuse by modulating the reward system and haemorhegic shock. Furthermore, MC3 and MC4 receptor agonists have antipyretic effects and both have been suggested to be involved in peripheral nerve regeneration and the MC4 receptor is also known to reduce stress response. In a further aspect, the invention relates to a method of activating MC4 in a subject, comprising administering to the subject an effective amount of a compound according to the invention.

In all therapeutic methods disclosed above, the compound of the present invention may be administered alone. However, as indicated above, it may also be administered in combination with one or more additional therapeutically active compounds, either sequentially or concomitantly.

In one aspect, the invention relates to a pharmaceutical composition comprising a compound of the present invention, optionally in combination with one or more additional therapeutically active compounds together with one or more pharmaceutically acceptable carrier or exipient in unit dosage form comprising about 0.05 mg to about 1000 mg, such as about 0.1 mg to about 500 mg, such as from about 0.5 mg to about 200 mg of a compound of the present invention.

The present invention also relates to the use of a compound of the present invention in the manufacture of a medicament for the treatment of a disease or state selected from overweight or obesity, bulimia, binge-eating, atherosclerosis, hypertension, type 2 diabetes, impaired glucose tolerance (IGT), dyspilidemia, coronary heart disease, gallbladder disease, gall stone, osteoarthritis, cancer (in particular endometrial, breast, colon and prostate cancer), sexual dysfunction and risk of premature death.

The present invention also relates to the use of a compound of the present invention, alone or in combination with an additional therapeutically active compound, in the manufacture of a medicament effective in delaying the progression from IGT to type 2 diabetes, delaying the progression from type 2 diabetes to insulin requiring diabetes, regulating appetite, inducing satiety, preventing weight gain after successfully having lost weight or increasing energy expenditure.

The invention thus relates, inter alia, to compounds of the general formula I:

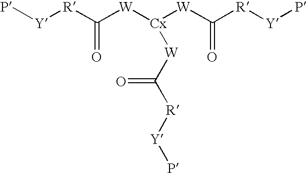

wherein Cx represents a center moiety having three independent arms;
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally, one or more groups B, said groups, when R′ contains one or more groups B, being linked in any order, with the proviso that that end of each R′ that is attached to the carbonyl group bound to W begins with a group A;
wherein A is

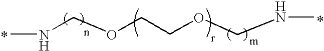

wherein r is 0 or an integer from 1 to 50,
and n and m independently are integers ≧2;

### B is

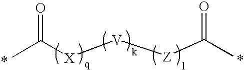

wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl, C₂₋₆-alkenyl, C₃₋₈-cycloalkyl and C₄₋₈-cycloalkenyl, or R¹ and R² taken together form a C₂₋₆-alkylene bridge;
R″ is selected from hydrogen, C₁₋₆-alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts, prodrugs and solvates thereof.

In an aspect of the invention the peptides P (which in compounds of the invention are covalently attached to Y, thereby forming P ‘Y’) independently are of the formula A:

R¹-X-X¹-X²-X³-X⁴-X⁵-X⁶-X⁷-X⁸-X⁹-X¹⁰-X¹¹-R² (formula A)

wherein R¹, which is bonded to an N-terminal NH₂-group, is either absent or represents C₁₋₄-alkanoyl or an anchoring group R^{X}, the anchoring group R^{X} optionally being attached to X via a linker S, the linker S, if present, being selected from β-alanine, Glu, Gly-Gln, Gly-Glu, Gly-His and

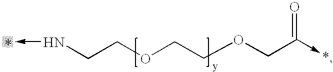

wherein y is 1, 2, 3, 4 or 5;
X represents a bond, an amino acid residue, or a di- or tri-peptide residue, wherein the amino acid(s) may be naturally occurring or synthetic;
X¹ represents a bond or an amino acid residue with a functional group in the side chain to which an anchoring group Rx may be attached, optionally via a linker S, the linker S, if present, being selected from β-alanine, Glu, Gly-Gln, Gly-Glu, Gly-His and

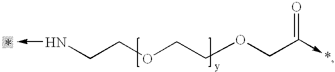

wherein y is 1, 2, 3, 4 or 5.

The anchoring group R^{x} may, for example, be a naturally occurring or synthetic amino acid residue.

In appropriate embodiments of the invention, the anchoring group Rx represents a group of formula X, Xa or Xb

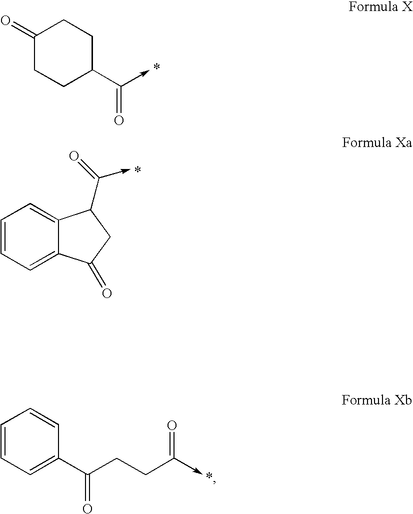

or R^{x} represents a C₂₋₆-alkanoyl or C₃₋₆-alkenoyl bearing a keto or aldehyde function which may react with the group Y to form Y′P′.
X² represents a bond, an amino acid residue, or a di-, tri- or tetra-peptide residue, wherein the amino acid(s) may be naturally occurring or synthetic;
X³ represents a bond or an amino acid residue, the amino acid residue optionally being capable of forming a bridge to X¹⁰;
X⁴ represents a bond, an amino acid residue or a di-peptide residue, wherein the amino acid(s) may be naturally occurring or synthetic;
X⁵ represents an amino acid residue selected from H is, Ala, Nle, Met, Met(O), Met(O₂), Gln, Gln(ε-alkyl), Gln(ε-aryl), Asn, Asn(ε-alkyl), Asn(ε-aryl), Ser, Thr, Cys, F-Pro, Pro, Hyp, (S)-1,2,3,4-tetrahydroisoquinoline-3-carboxylic acid, Trp, 1-naphthylalanine, 2-naphthylalanine, 2-pyridylalanine, 3-pyridylalanine, 4-pyridylalanine, 2-thienylalanine, 3-thienylalanine, 4-thiazolylalanine, 2-furylalanine, 3-furylalanine and Phe, wherein the phenyl moiety of said Phe is optionally substituted with halogen, hydroxyl, alkoxy, nitro, benzoyl, methyl, trifluoromethyl or cyano;
X⁶ represents D-Phe, wherein the phenyl moiety of said D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, an amino acid residue or a di-peptide residue, wherein the amino acid(s) may be naturally occurring or synthetic;
X¹⁰ represents a bond or an amino acid residue, said amino acid residue optionally being capable of forming a bridge to X³;
X¹¹ represents a bond, an amino acid residue or a di-peptide residue, wherein the amino acid(s) may be naturally occurring or synthetic;
R² represents —OH or —NR⁵R⁶, wherein R⁵ and R⁶ independently represent hydrogen, C₁₋₈alkyl, C₂₋₈alkenyl or C₂₋₈alkynyl;

Each P independently is optionally cyclized from X³ to X¹⁰ via a lactam or a disulfide bridge, In addition, each P comprises only one anchoring group, and each P comprises at least 7 amino acid residues.

As already indicated, the invention also relates to: the use of compounds of the formula I above in therapy; to pharmaceutical compositions comprising compounds of formula I; to methods of treatment comprising administering to a subject in need thereof an effective amount of a compound of formula I above; and to the use of compounds of formula I in the manufacture of medicaments.

### DEFINITIONS

The use of prefixes of the structure: C_{x-y}-alkyl, C_{x-y}-alkenyl, C_{x-y}-alkynyl or C_{x-y}-cycloalkyl designates a radical of the designated type having from x to y carbon atoms.

The terms “C₁₋₆-alkyl” or “C₁₋₆-alkylene” refer to a saturated, branched or straight hydrocarbon group having from 1 to 6 carbon atoms. Typical C₁₋₆-alkyl groups include, but are not limited to, methyl, ethyl, n-propyl, isopropyl, butyl, isobutyl, sec-butyl, tert-butyl, pentyl, hexyl and the corresponding divalent radicals.

The terms “C₂₋₆-alkenyl” or “C₂₋₆-alkenylene” refer to a branched or straight hydrocarbon group having from 2 to 6 carbon atoms and at least one double bond. Typical C₂₋₆-alkenyl groups include, but are not limited to, ethenyl, 1-propenyl, 2-propenyl, isopropenyl, 1,3-butadienyl, 1-butenyl, 2-butenyl, 1-pentenyl, 2-pentenyl, 1-hexenyl, 2-hexenyl, 1-ethylprop-2-enyl, 1,1-(dimethyl)prop-2-enyl, 1-ethylbut-3-enyl, 1,1-(dimethyl)but-2-enyl, and the corresponding divalent radicals.

The terms “C₂₋₆-alkynyl” or “C₂₋₆-alkynylene” refer to a branched or straight hydrocarbon group having from 2 to 6 carbon atoms and at least one triple bond. Typical C₂₋₆-alkynyl groups include, but are not limited to, vinyl, 1-propynyl, 2-propynyl, isopropynyl, 1,3-butadynyl, 1-butynyl, 2-butynyl, 1-pentynyl, 2-pentynyl, 1-hexynyl, 2-hexynyl, 1-ethylprop-2-ynyl, 1,1-(dimethyl)prop-2-ynyl, 1-ethylbut-3-ynyl, 1,1-(dimethyl)but-2-ynyl, and the corresponding divalent radicals.

The terms “C₁₋₆-alkyloxy”, “C₁₋₆-alkyleneoxy”, “C₂₋₆-alkenyloxy”, “C₂₋₆-alkenyleneoxy”, “C₂₋₆-alkynyloxy” or “C₂₋₆-alkynyleneoxy”; refer to the radical —O—C₁₋₆-alkyl, —O—C₁₋₆-alkylene, —O—C₂₋₆-alkenyl, —O—C₂₋₆-alkenylene, —O—C₂₋₆-alkynyl or —O—C₂₋₆-alkynylene wherein C₁₋₆-alkyl(ene), C₂₋₆-alkenyl or C₂₋₆-alkynyl are as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “halogen” is intended to indicate fluoro, chloro, bromo and iodo.

In the context of this invention the term “-triyl” is used and refers to different alkyl, alkenyl, alkynyl, cycloalkyl or aromatic radicals with three attachment points.

The terms “C₃₋₈-cycloalkyl” or “C₃₋₈-cycloalkylene” refer to a monocyclic, carbocyclic group having from 3 to 8 carbon atoms or the corresponding biradical. Representative examples are cyclopropyl, cyclobutyl, cyclopentyl, cyclohexyl, cycloheptyl, cyclooctyl, and the like.

The terms “C₄₋₈-cycloalkenyl” or “C₄₋₈-cycloalkenylene” refer to C₄₋₈-cycloalkenyl representing a monocyclic, carbocyclic, non-aromatic group having from 4 to 8 carbon atoms or the corresponding biradical and at least one double bond. Representative examples are cyclobutenyl, cyclopentenyl, cyclohexenyl, cycloheptenyl, cyclooctenyl and the like.

The terms “aryl” or “arylene” as used herein are intended to include carbocyclic aromatic ring systems such as phenyl, biphenylyl, naphthyl, anthracenyl, phenanthrenyl, fluorenyl, indenyl, pentalenyl, azulenyl and the like and the corresponding biradicals (e.g. o-, m- and p-phenylene). Aryl or Arylene are also intended to include the partially hydrogenated derivatives of the carbocyclic systems. Non-limiting examples of such partially hydrogenated derivatives are 1,2,3,4-tetrahydronaphthyl, 1,4-dihydronaphthyl and the like.

The terms “heteroaryl” or “heteroarylene” as used herein are intended to include heterocyclic aromatic ring systems containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like and the corresponding biradicals. Heteroaryl or heteroarylene are also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The terms “heterocyclyl” or “hetereocycylene as used herein denote the fully hydrogenated derivatives of the heteroaryls or the heteroarylenes, respectively, defined above.

The term heteroaryl-C₁₋₆-alkyl as used herein denotes heteroaryl as defined above and C₁₋₆-alkyl as defined above.

The terms “aryl-C₁₋₆-alkyl” and “aryl-C₂₋₆-alkenyl” as used herein denote aryl as defined above and C₁₋₆-alkyl and C₂₋₆-alkenyl, respectively, as defined above.

The term “acyl” as used herein denotes —(C═O)—C₁₋₆-alkyl wherein C₁₋₆-alkyl is as defined above.

The terms “C₂₋₆-alkanoyl” or “C₂₋₆-alkanoylene” as used herein denote —(C═O)—C₁₋₅-alkyl or —(C═O)—C₁₋₅-alkylene, respectively.

The terms “C₃₋₆-alkenoyl” or “C₃₋₆-alkenoylene” as used herein denote —(C═O)—C₂₋₅-alkenyl or —(C═O)—C₂₋₅-alkenylene, respectively.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

As used herein, the term “solvate” refers to a complex of defined stoichiometry formed between a solute (in casu, a compound according to the present invention) and a solvent. Solvents may include, by way of example, water, ethanol, or acetic acid. A solvate wherein water is the solvent in question is often referred to as a “hydrate”, and such hydrates are thus within the scope of the present invention.

In addition, the following abbreviations have the indicated meanings:

| | |
|---|---|
| Ac | Acetyl |
| 4-Abu | 4-aminobutyric acid |
| Ala | Alanine |
| Arg | Arginine |
| Arg(Pmc) | 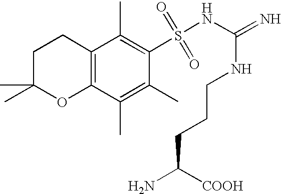 |
| Asn | Asparagines |
| Asn(alkyl) | 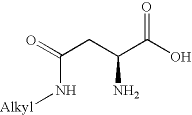 |
| Asn(aryl) | 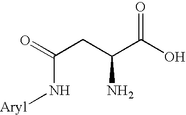 |
| Asp | aspartic acid |
| Boc | tert-butyloxycarbonyl |
| Cys | Cysteine |
| D-Phe | D form of phenylalanine |
| Fmoc | 9-fluorenylmethoxycarbonyl |
| F-Pro | 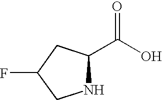 |
| Gly | Glycine |
| Gln | Glutamine |
| Gln(alkyl) | 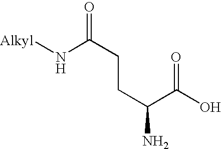 |
| Gln(aryl) | 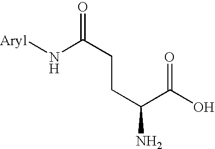 |
| Glu | glutamic acid |
| His | Histidine |
| homoArg | 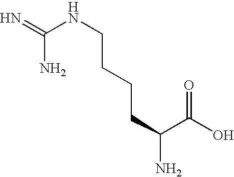 |
| homoCys | 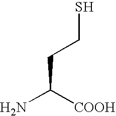 |
| Hyp | 4-hydroxyproline |
| Ile | Isoleucine |
| Leu | Leucine |
| Lys | Lysine |
| Met | Methionine |
| Met(O) | 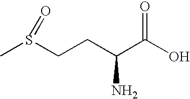 |
| Met(O₂) | 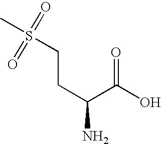 |
| mPEG2000 | methoxypolyethylenglycol (average molecular weight of |
| | 2000 Dalton) |
| Mtt | 4-methyltrityl |
| 2NaI | 2-Naphthylalanine |
| Nle | Norleucine |
| Orn | Ornithine |
| Phe | Phenylalanine |
| Pro | Proline |
| 2-PyAla | 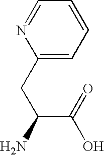 |
| 3-PyAla | 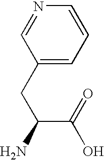 |
| 4-PyAla | 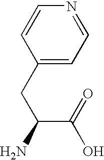 |
| Ser | Serine |
| Thr | Threonine |
| Tic | (S)-1,2,3,4-tetrahydroisoquinoline-3-carboxylic acid |
| tBu | tert-butyl |
| Thr | Threonine |
| Trp | Tryptophan |
| Val | Valine |

### —CH₂(OCH₂CH₂)₁₋₆OCH₂— or —CH₂OCH₂—;

In another aspect, X, V and Z taken together represent —(CH₂)₃— or —CH₂OCH₂—

In further aspects of the invention, B has one of the following structures:

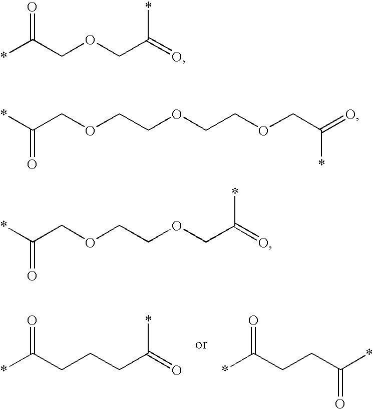

In an aspect of the invention, X, V and Z independently represent arylene or heteroarylene.

In one aspect, V is o-, m- or p-phenylene.

In one aspect, V is C₃₋₈-cycloalkylene or C₃₋₈-cycloalkenylene.

In another aspect, V is a cyclopentylene, a cyclohexylene or a cyclohexenylene.

In yet another aspect, V is a diradical derived from morpholine, piperazine, dioxan or thiophene.

The group Y″, which can be defined as the monomeric building block leading to Y in the formula above and to Y′ when attached to a peptide, has two reactive functional groups. It reacts with either group A or group B depending on the characteristics of the R′ when assembling the trimerisation molecule. Thus, if R′ has been chosen to contain a B as the last constituent of the R′, Y″ should contain a reactive functional group which can be attached to an acid derivative. If the R′ has been chosen to contain an A group as the last constituent of the R′, Y″ should contain a reactive functional group suitable for reacting with amines.

Additional abbreviations employed herein include Dab [(S)-2,4-diaminobutyric acid] and Dap [(S)-2,3-diaminopropionic acid].

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist.

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

### DESCRIPTION OF THE INVENTION

In an aspect of the invention the peptide is assembled on solid phase and selected amino acids are substituted with amino acids with suitable side chains acting as attachment groups, using standard solid phase chemistry. Examples of such amino acid substitutions are by way of non-limiting illustration: substitution of serine with cystein, substitution of phenylalanine with tyrosine, or substitution of arginine with lysine. Alternatively, attachment groups are introduced by enzyme directed coupling in either the C- or N-terminal end of the peptide, with either suitable amino acids allowing for post modificational attachment of polymers, or small organic molecules serving the same purpose. Enzymes that support this aspect of the invention include by way of non-limiting illustration: carboxypeptidases, and proteases in reverse.

In one embodiment of compounds of the invention, all three peptides P (which form P′ in P′Y′) are identical.

### Cyclic Peptides of Formula A

A peptide P may be cyclic or non-cyclic. In one embodiment, there is a bridge between X³ and X¹⁰ in a peptide P, rendering the peptide, and thus the compound of formula I, cyclic, either by the presence of a disulfide bridge formed between X³ and X¹⁰ moieties independently selected from Cys and homoCys, or by the presence of a lactam bond formed between a carboxylic acid moiety in the side-chain of X³ and an amine moiety in the side-chain of X¹⁰, or between a carboxylic acid moiety in the side-chain of X¹⁰ and an amine moiety in the side-chain of X³.

In one embodiment, X in formula A is a bond.

In one embodiment, X¹ in formula A represents a bond.

In one embodiment, X² in formula A represents Nle.

In one embodiment, X³ in formula A represents Glu or Asp, and X¹⁰ represents Lys, Orn, Dab or Dap. In another embodiment, X³ represents Glu or Asp, and X¹⁰ represents Lys. In a further embodiment, X³ represents Glu, and X¹⁰ represents Lys.

In one embodiment, X⁴ represents a bond.

In one embodiment, X⁵ represents His. In another embodiment, X⁵ represents 3-PyAla, Hyp, Gln or Asn.

In one embodiment, X⁹ represents a bond.

In one embodiment, X¹¹ represents a bond.

In one embodiment, R² represents —NH₂

In one embodiment, X⁶-X⁷-X⁸-X⁹-X¹⁰ represents D-Phe-Arg-Trp-Lys.

### Non-Cyclic Peptides of Formula A

In one embodiment, the compound of formula A, and thus the compound of formula I, I is non-cyclic.

In one embodiment, X represents a bond.

In one embodiment, X represents an amino acid residue, such as e.g. Ser.

In one embodiment, X¹ represents Lys(N^{ε}β-Ala-R^{X}).

In one embodiment, X¹ represents a bond.

In one embodiment, X² represents Tyr-Ser-Nle.

In one embodiment, X² represents Ser-Nle.

In one embodiment, X² represents Ser-Tyr-Ser-Nle.

In one embodiment, X³ represents Glu.

In one embodiment, X⁴ represents a bond.

In one embodiment, X⁵ represents His.

In one embodiment, X⁵ represents Gln, Hyp, 3-PyAla, Ala or Ser.

In one embodiment, X⁹ represents Gly.

In one embodiment, X¹⁰ represents Lys or Arg.

In one embodiment, X¹¹ represents Pro-Val.

In one embodiment, R² represents —NH₂.

In further embodiments, X⁶-X⁷-X⁸-X⁹-X¹⁰ represents D-Phe-Arg-Trp-Lys.

In an aspect of the invention, a compound of the invention is represented by a formula among the following:

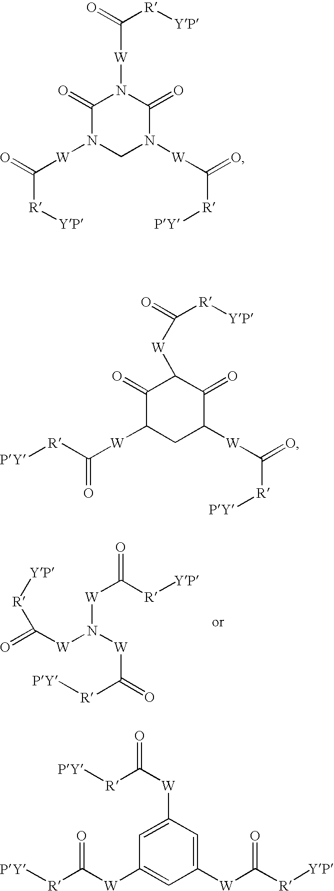

wherein R′, W and Y′ and P′ are as defined above.

In an aspect of the invention, W is a bond or C₁₋₆-alkylene;

In a further aspect of the invention, a compound of the invention is more specifically represented by one of the formulas below:

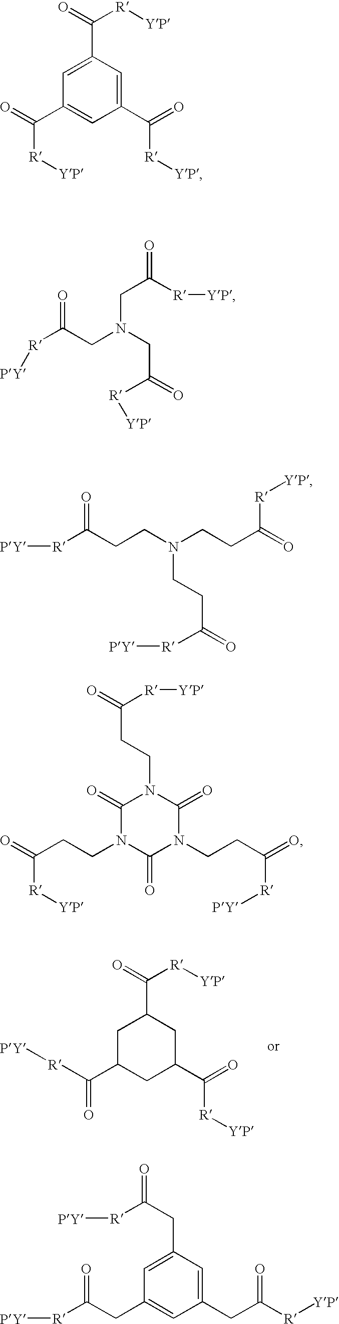

wherein R′ and Y′ and P′ are as defined above.

In an aspect of the invention, the parameter r in A in formula I is 0 or an integer from 1 to 25; n and m are as defined above. The parameter r is suitably in the range 0-10, such as 0-5 (i.e. 0, 1, 2, 3, 4 or 5), e.g. 0-3 (i.e. 0, 1, 2, or 3).

In an aspect of the invention r is 0, 1, 2 or 3;

In an aspect of the invention n and m are below 15. In an aspect of the invention n and m are below 10; In an aspect of the invention n and m are below 6. In an aspect of the invention n and m are independently selected from 2, 3, 4 or 5. In an aspect of the invention n and m are independently selected from 2 or 3;

In an aspect of the invention A is any of the structures below:

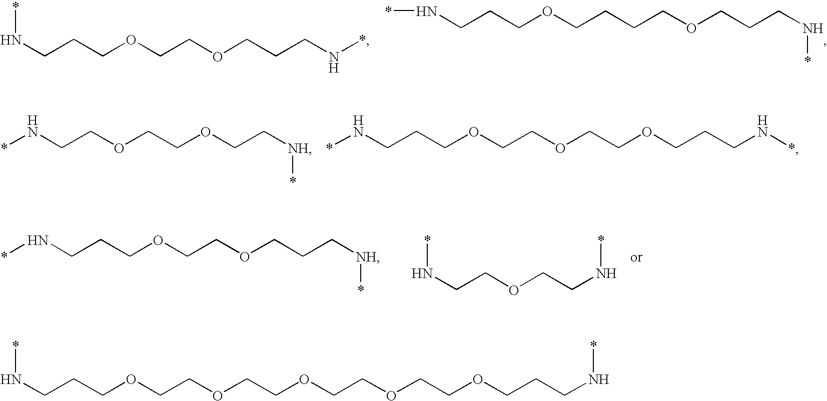

In another aspect of the invention, X, V and Z in an element B in R′ in formula I independently represent —CR¹R²—, —O—, —S—, —NR″—, —OCH₂CH₂O—, —OCH₂CH₂—, —CH₂CH₂O—, —OCH₂— or —CH₂O—, wherein R¹, R² and R″ are as defined above.

In one aspect, X, V and Z independently represent —CR¹R²—, wherein R¹ and R² are as defined above.

In another aspect, X, V and Z independently represent —OCH₂CH₂O—, —OCH₂— or —CH₂O—.

In a further aspect, X, V and Z taken together represent —(CH₂)₁₋₄—,

The other functionality of Y″ and Y should be able to react with peptides; this could, for example, be a side-chain of a naturally occurring peptide, some of which contain appropriate functional groups, or a moiety attached to an amino acid either internally in the sequence or at the N- or C-terminal.

The term “reactive functional group” designates, by way of illustration and without limitation, any free amino, carboxyl, thiol, alkyl halide, acyl halide, chloroformiate, aryloxycarbonate, hydroxy or aldehyde group, carbonates such as the p-nitrophenyl, or succinimidyl; carbonyl imidazoles, carbonyl chlorides; carboxylic acids that are activated in situ; carbonyl halides, activated esters such as N-hydroxysuccinimide esters, N-hydroxybenzotriazole esters, esters such as those comprising 1,2,3-benzotriazin-4(3H)-one, phosphoramidites and H-phosphonates, phosphotriesters or phosphodiesters activated in situ, isocyanates or isothiocyanates, in addition to groups such as —NH₂, —OH, —N₃, —NHR′, —OR′, —ONH₂, alkynes, or any of the following:

| | |
|---|---|
| hydrazine derivatives | ——NH—NH₂, |
| hydrazine carboxylate | —O—C(O)—NH—NH₂, |
| derivatives | |
| semicarbazide derivatives | —NH—C(O)—NH—NH₂, |
| thiosemicarbazide | —NH—C(S)—NH—NH₂, |
| derivatives | |
| carbonic acid dihydrazide | —NHC(O)—NH—NH—C(O)—NH—NH₂, |
| derivatives | |
| carbazide derivatives | —NH—NH—C(O)—NH—NH₂, |
| thiocarbazide derivatives | —NH—NH—C(S)—NH—NH₂, |
| aryl hydrazine derivatives | —NH—C(O)—C₆H₄—NH—NH₂, |
| hydrazide derivatives | —C(O)—NH—NH₂; |
| and | |
| oxylamine derivatives, | —C(O)—O—NH₂, |
| such as | —NH—C(O)—O—NH₂ and |
| | —NH—C(S)—O—NH₂. |

Other functional groups present may be suitably protected by protection groups. Appropriate protection groups are known to the skilled person, and examples can be found in Green & Wuts “Protection groups in organic synthesis”, 3.ed. Wiley-interscience.

Thus in an aspect of the invention, each Y independently is a structure as follows:

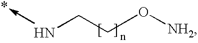

wherein n is an integer ≧0.

In a further aspect, all three Y groups have the following structure:

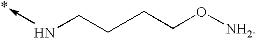

In an aspect of the invention, the structure of each group R′ independently is selected among the following:

### -A-, -A-B-, -A-B-A-, -A-B-A-B-, -A-B-A-B-A- and -A-B-A-B-A-B-.

In one aspect, all three R′ groups are identical.

In another aspect, two of the three R′ groups are identical.

In a further aspect, three different R′ groups are present.

A further aspect of the invention provides a compound according to the invention (compound of formula I) for use in therapy.

According to the present invention, the trimerisation molecules are assembled according to the methods described, after which the peptides are coupled. Trimerisation molecules having a maleimide moiety are coupled to peptides via a Michael reaction with a free cysteine on the peptide. Trimerisation molecules having an aminooxy moiety are coupled to peptides via the formation of an oxime with a keto or aldehyde group on the peptide. The peptides may contain reactive functional groups, such as amines, thiols, keto groups, aldehyde groups or hydroxyl groups, which react with the group Y.

### Pharmaceutical Compositions

The compounds for use according to the present invention may be administered alone or in combination with pharmaceutically acceptable carriers or excipients, in either single or multiple doses. The pharmaceutical compositions according to the present invention may be formulated with pharmaceutically acceptable carriers or diluents as well as any other known adjuvants and excipients in accordance with conventional techniques such as those disclosed in Remington: The Science and Practice of Pharmacy, 20^{th} Edition, Gennaro, Ed., Mack Publishing Co., Easton, Pa., 2000.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the parenteral and sublingual routes being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typically doses are in the order of about half the dose employed for oral administration.

The compounds for use according to the present invention are generally utilized as the free substance or as a pharmaceutically acceptable salt thereof. Examples are an acid addition salt of a compound having the utility of a free base and a base addition salt of a compound having the utility of a free acid. The term “pharmaceutically acceptable salts” refers to non-toxic salts of the compounds for use according to the present invention which are generally prepared by reacting the free base with a suitable organic or inorganic acid or by reacting the acid with a suitable organic or inorganic base. When a compound for use according to the present invention, such as a compound of Formula (I), contains a free base such salts are prepared in a conventional manner by treating a solution or suspension of the compound with a chemical equivalent of a pharmaceutically acceptable acid. When a compounds for use according to the present invention, such as a compound of Formula (I), contains a free acid such salts are prepared in a conventional manner by treating a solution or suspension of the compound with a chemical equivalent of a pharmaceutically acceptable base. Physiologically acceptable salts of a compound with a hydroxy group include the anion of said compound in combination with a suitable cation such as sodium or ammonium ion. Other salts which are not pharmaceutically acceptable may be useful in the preparation of compounds for use according to the present invention and these form a further aspect of the present invention.

For parenteral administration, solutions of the compounds for use according to the present invention in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds for use according to the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents, and preserving agents in order to provide pharmaceutically elegant and palatable preparations. Tablets may contain the active ingredient in admixture with non-toxic pharmaceutically-acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,356,108; 4,166,452; and 4,265,874, incorporated herein by reference, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsule wherein the active ingredient is mixed with water or an oil medium, for example peanut oil, liquid paraffin, or olive oil.

Aqueous suspensions may contain the active compounds in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising a compound for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the present invention. These compositions can be prepared by mixing the drug with a suitable non-irritating excipient which is solid at ordinary temperatures but liquid at the rectal temperature and will thus melt in the rectum to release the drug. Such materials include cocoa butter and polyethylene glycols, for example.

For topical use, creams, ointments, jellies, solutions of suspensions, etc., containing the compounds of the present invention are contemplated. For the purpose of this application, topical applications shall include mouth washes and gargles.

The compounds for use according to the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some of the compounds for use according to the present invention may form solvates with water or common organic solvents. Such solvates are also encompassed within the scope of the present invention.

Thus, in a further embodiment, there is provided a pharmaceutical composition comprising a compound for use according to the present invention, or a pharmaceutically acceptable salt, solvate, or prodrug thereof, and one or more pharmaceutically acceptable carriers, excipients, or diluents.

If a solid carrier is used for oral administration, the preparation may be tableted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

### Dosage and Combination Therapies

A typical dosage of a compound of the invention when employed in accordance with the present invention is in the range of from about 0.001 to about 100 mg/kg body weight per day, e.g. from about 0.01 to about 50 mg/kg body weight per day, such as from about 0.01 to about 10 mg/kg body weight per day, e.g. from about 0.01 to about 1 mg/kg body weight per day, administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

As already mentioned, the invention relates, inter alia, to a pharmaceutical composition comprising a compound of the present invention, optionally in combination with one or more additional therapeutically active compounds or substances, together with one or more pharmaceutically acceptable carriers or excipients. The composition may suitably be in unit dosage form comprising from about 0.05 mg to about 1000 mg, such as from about 0.1 mg to about 500 mg, e.g. from about 0.5 mg to about 200 mg, of a compound of the present invention, and a typical unit dosage form intended for oral administration one or more times per day, such as from one to three times per day, may suitably contain such amounts, i.e. from 0.05 to about 1000 mg, e.g. from about 0.1 to about 500 mg, such as from about 0.5 mg to about 200 mg of a compound of the invention.

In all of the therapeutic methods disclosed herein, a compound of the present invention may be administered alone or in combination with one or more (i.e. one or two or three . . . etc.) additional compounds of the present invention. Moreover, a compound of the invention, or a combination of two or more (i.e. two or three or four . . . etc.) compounds of the invention, may be administered in combination with one or more other therapeutically active agents or compounds (i.e. agents or compounds which are not within the scope of the present invention), either sequentially or concomitantly.

Suitable additional therapeutic compounds, agents or substances may be selected, for example, from antidiabetic agents, antihyperlipidemic agents, antiobesity agents, antihypertensive agents and agents for the treatment of complications resulting from, or associated with, diabetes.

Suitable antidiabetic agents include: insulin; derivatives or analogues of insulin, including derivatives or analogues exhibiting a profile of protracted or prolonged activity, such as those disclosed in WO 95/07931, WO 97/31022 and WO 2005/012347 (Novo Nordisk A/S), the contents of all of which are incorporated herein by reference; derivatives of GLP-1 (glucagon like peptide-1), such as those disclosed in WO 98/08871 (Novo Nordisk A/S), the contents of which are incorporated herein by reference; derivatives of GLP-1 analogues, such as those disclosed in U.S. Pat. No. 6,458,924 (Knudsen et al.), the contents of which are incorporated herein by reference; and orally active hypoglycemic agents.

Suitable orally active hypoglycemic agents include: imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk A/S), the contents of which are incorporated herein by reference; potassium channel openers such as ormitiglinide; potassium channel blockers such as nateglinide or BTS-67582; glucagon antagonists such as those disclosed in WO 99/01423 and WO 00/39088 (Novo Nordisk A/S and Agouron Pharmaceuticals, Inc.), the contents of which are incorporated herein by reference; GLP-1 agonists such as those disclosed in WO 00/42026 (Novo Nordisk A/S and Agouron Pharmaceuticals, Inc.), the contents of which are incorporated herein by reference; DPP-IV (dipeptidyl peptidase-IV) inhibitors; PTPase (protein tyrosine phosphatase) inhibitors; glucokinase activators, such as those described in WO 2004/002481 (Novo Nordisk), the contents of which are incorporated herein by reference, and in WO 02/08209 (Hoffmann La Roche); inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as antihyperlipidemic agents and antilipidemic agents; compounds lowering food intake; as well as PPAR (peroxisome proliferator-activated receptor) agonists and RXR (retinoid X receptor) agonists such as ALRT-268, LG-1, 268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-1037 or T 174, or the compounds disclosed in WO 97/41097 (DRF-2344), WO 97/41119, WO 97/41120, WO 00/41121 and WO 98/45292 (Dr. Reddy's Research Foundation), the contents of all of which are incorporated herein by reference.

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), the contents of all of which are incorporated herein by reference.

Still further examples of suitable additional therapeutically active substances include:

α-glucosidase inhibitors, e.g. voglibose, emiglitate, miglitol or acarbose;
glycogen phosphorylase inhibitors, e.g. the compounds described in WO 97/09040 (Novo Nordisk A/S);
glucokinase activators; and
agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y) antagonists, Y2 and Y4 receptor agonists, MC3 (melanocortin 3) agonists, MC3 (melanocortin 3) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds (growth hormone secretagogues), ghrelin antagonists, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, the contents of all of which are incorporated herein by reference, exendin-4, GLP-1 agonists and ciliary neurotrophic factor.

Further suitable antiobesity agents are bupropion (antidepressant), topiramate (anticonvulsant), ecopipam (dopamine D1/D5 antagonist), naltrexone (opioid antagonist), and peptide YY₃₋₃₆ (Batterham et al, Nature 418, 650-654 (2002)).

An embodiment of a suitable antiobesity agent for use in a method of the invention as an additional therapeutically active substance in combination with a compound of the invention is leptin.

A further embodiment of a suitable antiobesity agent is peptide YY₃₋₃₆.

Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

In certain embodiments of the uses and methods of the present invention, the compound of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

### EXAMPLES

### List of Abbreviations Employed

AcOEt ethyl acetate
DCM dichloromethane
DIC diisopropylcarbodiimide
DIPEA ethyldiisopropylamine
DMAP 4-N,N-dimethylaminopyridine
DMEM Dulbecco's Modified Eagle's Medium
DMF N,N-dimethylformamide
DMSO dimethyl sulfoxide
EGTA 1,2-di(2-aminoethoxy)ethane-N,N,N′,N′-tetraacetic acid
FCS fetal calf serum
Fmoc 9-fluorenylmethyloxycarbonyl
HBTU 2-(1H-Benzotriazole-1-yl)-1,1,3,3-tetramethyluronium hexafluorophosphate
HEPES 2-[4-(2-hydroxyethyl)-piperazin-1-yl]-ethanesulfonic acid
HOAt 1-hydroxy-7-azabenzotriazole
HOBt 1-hydroxybenzotriazole
HSA human serum albumin
MeCN acetonitrile
α-MSH α-form of melanocyte-stimulating hormone
Mtt 4-methyltrityl
MTX methotrexate
NMP N-methylpyrrolidone
Pbf 2,2,4,6,7-pentamethyldihydro-benzofuran-5-sulfonyl
PBS phosphate-buffered saline
PEI polyethyleneimine
PyBop (benzotriazol-1-yloxy)tripyrrolidino-phosphonium hexafluorophosphate
TFA trifluoroacetic acid

### General Methods of Preparation

### General Procedure (A)

### Step A:

The molecules required for the trimerization of peptides are build up from amines and acids via the formation of an amide bond. In the case there is involved a di- or trifunctional molecule in which only one group is to react, the other groups can be protected using standard protecting groups.

The synthesis starts with the mono protection of the diamine building block, which is represented by A in the general formula, using procedures known to those skilled in the art (e.g. T. W. Greene, P. G. M. Wuts, Protective groups in organic synthesis, 2^{nd} ed., 1991 John Wiley & Sons, Inc. New York).

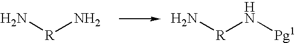

The diacid building block, represented by B in the general formula, can be protected using procedures known to those skilled in the art (e.g. T. W. Greene, P. G. M. Wuts, Protective groups in organic synthesis, 2^{nd} ed., 1991 John Wiley & Sons, Inc. New York).

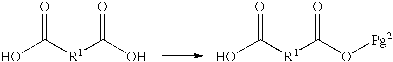

### Step B:

The next step involves the amide bond formation between the different building blocks. E.g. the central trifunctional acid is coupled with three mono protected diamines using standard amide bond formation conditions known to those skilled in the art. Preferable the acid is activated using a carbodiimde like e.g. diisopropylcarbodiimde and an active ester is formed e.g. a benzotriazoyl ester. The active ester is then reacted with the monoprotected diamine:

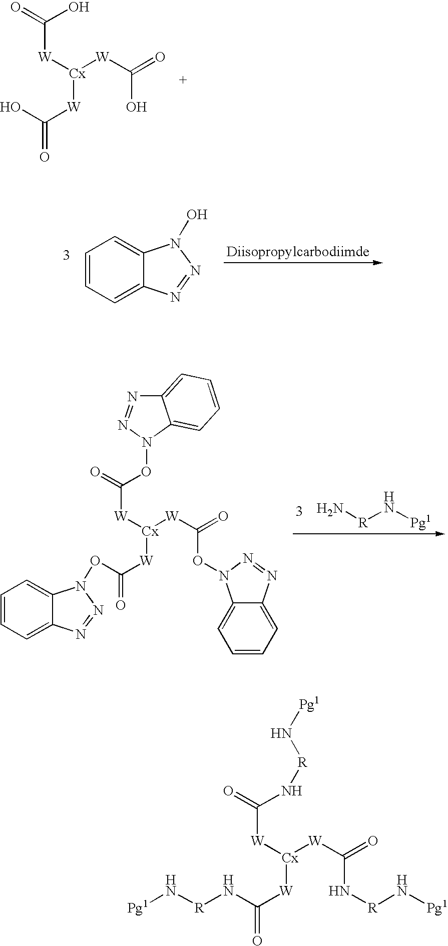

The reaction between an active ester and an amine can be promoted by the addition of a tertiary amine, e.g. diisoproylethylamin or triethylamine.

The protecting groups are removed with standard methods known to those skilled in the art (e.g. T. W. Greene, P. G. M. Wuts, Protective groups in organic synthesis, 2^{nd} ed., 1991 John Wiley & Sons, Inc. New York) before the triamine is allowed to react further with a mono protected mono activated diacid, B, or an activated acid containing a moiety (e.g. β-maleimido propionic acid) which terminates the arm and allows the molecule to react with a functional group on the peptide or protein, referred to as Y in the general formula. The reaction below demonstrates the incorporation of a C moiety:

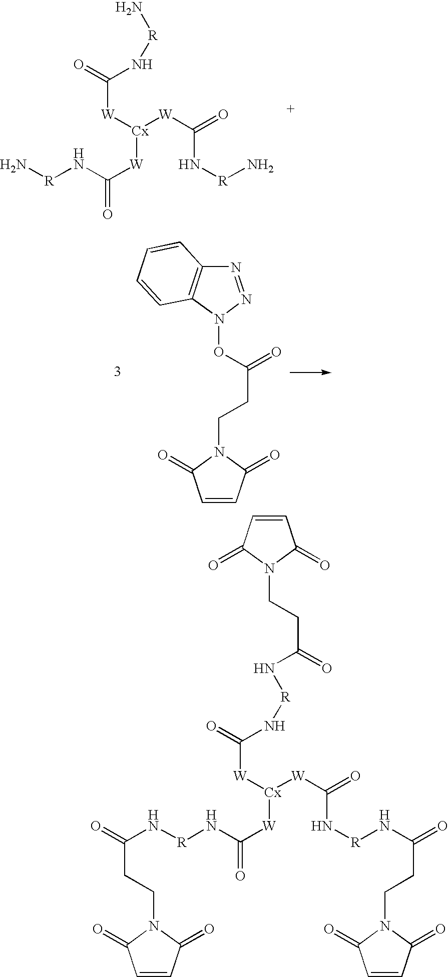

The activation and reaction of the acid is known to those skilled in the art and has be described earlier in this procedure. Prior to setting the terminating group on the arms, the arms can be elongated by subsequent attachments of diacids, B, and diamines, A, using the procedure here described.

General Procedure (B)

A sulfanyl-pyrrolidine-2,5-dione moiety may be formed by dissolving the peptide containing a thiol in question in water. Organic solvents may be added to increase solubility.

The solution is buffered to a suitable pH-value such as e.g. between pH 0 and pH 10, between pH 3 and pH 6, or pH 5 and kept at a suitable temperature such as e.g. 0-60° C. The maleimide in question is added, and the sulfanyl-pyrrolidine-2,5-dione moiety is formed according to the reaction scheme below:

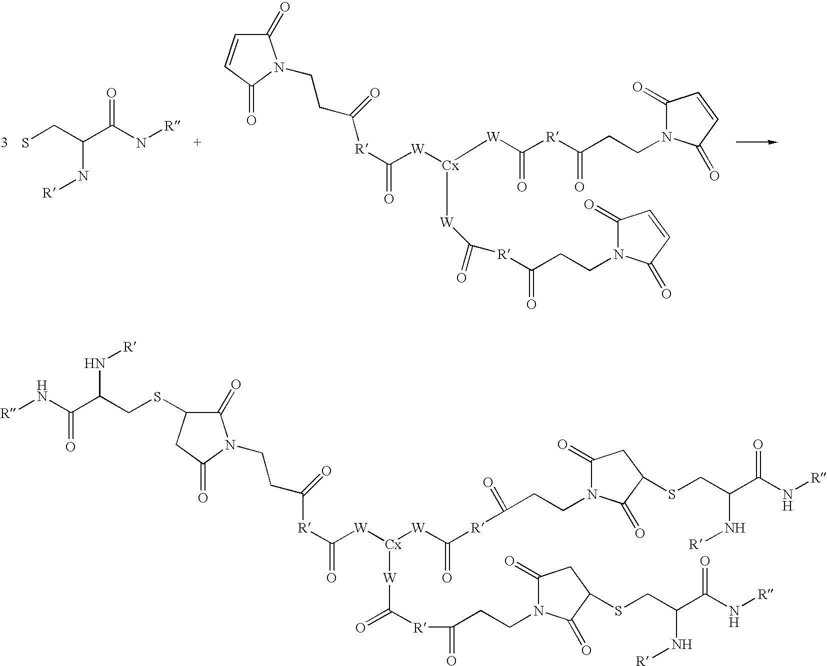

The pH of choice is determined e.g. by the solubility of the peptide to be. Solubility of peptides is to a large extent determined by the pKa of the peptide. Normally, the solubility of a given peptide is at its minimum when pH equals pKa of the peptide. At pH values between 7-14 other nucleophilic amino acid sidechains can react with the maleimde moiety and it lies within the skills of a skilled person to select a pH at which to run the reaction taking due care to the above consideration.

### General Procedure (C)

An oxime moiety may be formed by dissolving the peptide in question which contains a keto or aldehyde functionality, in water. Organic solvents may be added to increase solubility. The solution is buffered to a suitable pH-value such as e.g. between pH 0 and pH 10, between pH 3 and pH 6, or pH 5 and kept at a suitable temperature such as e.g. 0-60° C. The hydroxylamine in question is added, and the oxime moiety is formed according to the reaction scheme below:

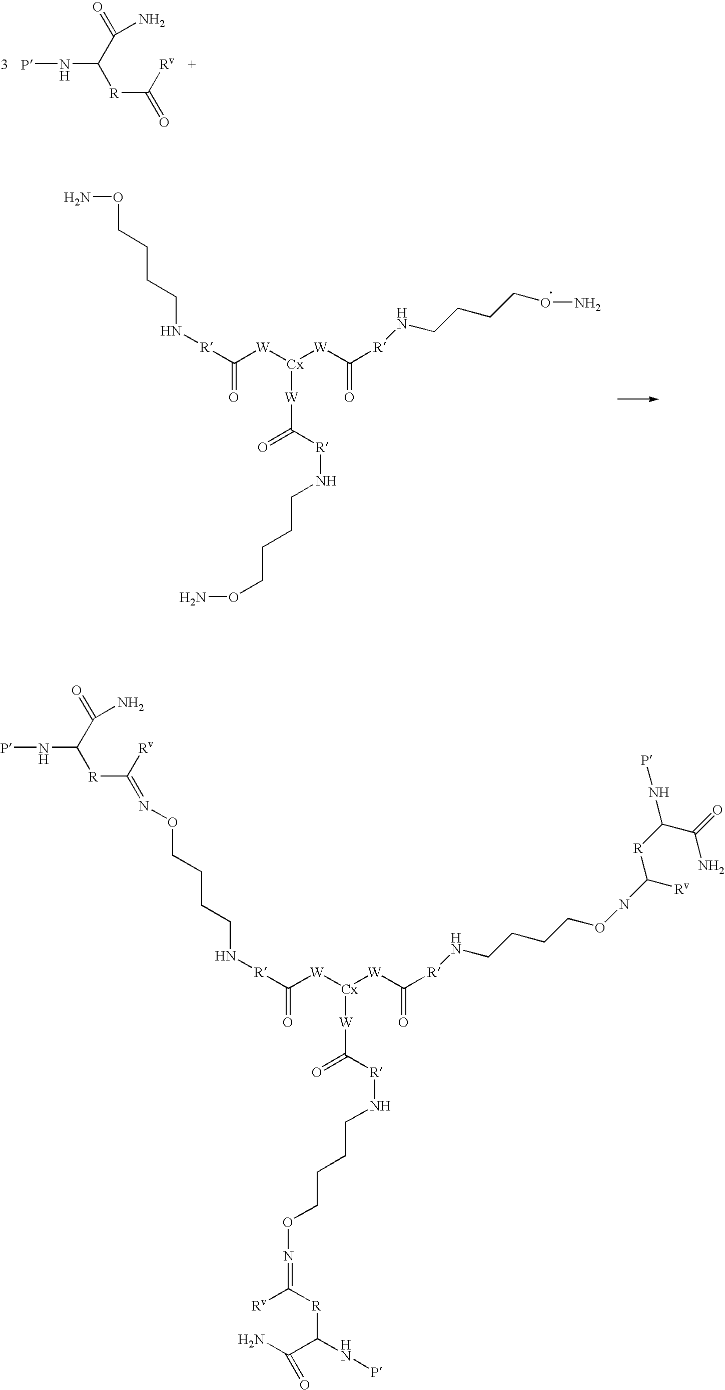

The pH of choice is determined e.g. by the solubility of the peptide to be. Solubility of peptides is to a large extent determined by the pKa of the peptide. Normally, the solubility of a given peptide is at its minimum when pH equals pKa of the peptide. It lies within the skills of a skilled person to select a pH at which to run the reaction taking due care to the above considerations.

All compounds of the present can be synthesized by those skilled in the art using standard coupling and deprotection steps. A description of all necessary tools and synthetic methods can be found in “The Fine Art Of Solid Phase Synthesis”, 2002/3 Catalog, Novabiochem.

Typical examples which include a cyclization step are as follows:

### Example A

### N{4-[4-(2-{[3-(2-{2-[3-(acetylamino)-propoxy]-ethoxy}-ethoxy)-propylcarbamoyl]-methoxy}-acetylamino)-butoxyimino]-cyclohexanecarbonyl-Gly-Ser-Gln-His-Ser-Nle-c[Glu-Hyp-D-Phe-Arg-Trp-Lys]-NH₂}₃

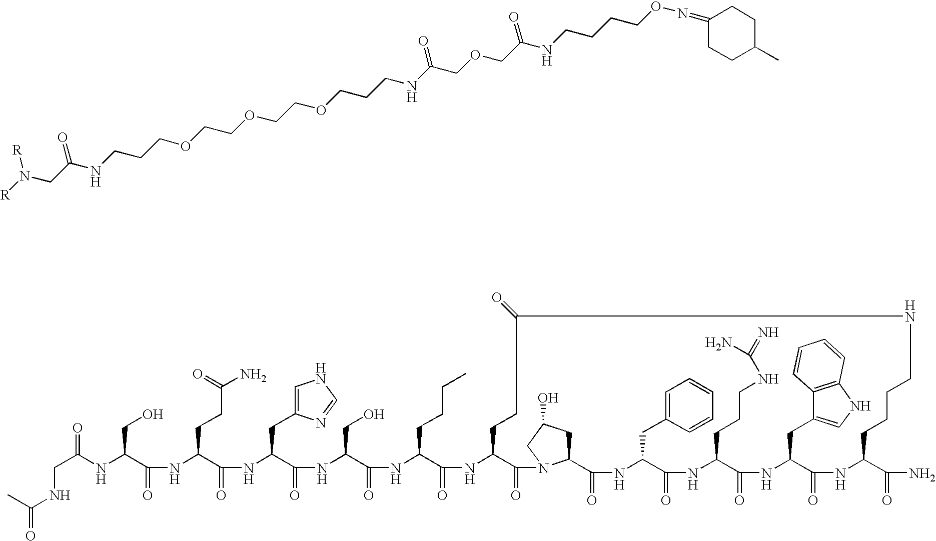

In the drawing the two R represent the other two arms of the trimer, which have been collapsed in order to be able to show one arm in a resonable scale.

Synthesis of the Peptide

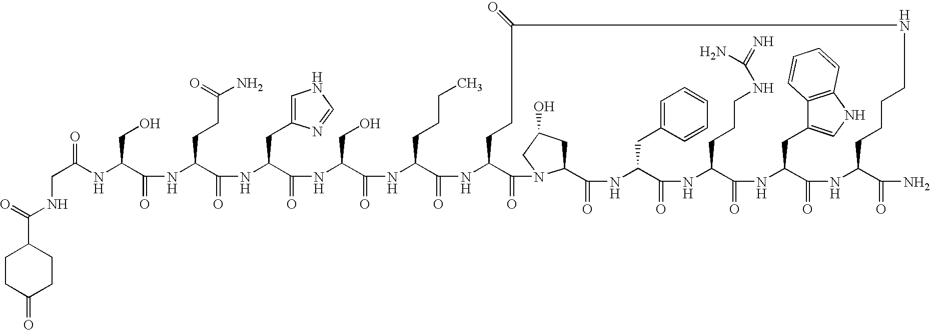

1.a. The protected peptidyl resin 4-oxocyclohexanecarbonyl-Gly-Ser(tBu)-Gln(Trt)-His(Trt)-Ser(tBu)-Nle-Glu(2-phenylisopropyloxy)-Hyp(tBu)-D-Phe-Arg(Pmc)-Trp(Boc)-Lys(Mtt)-(Rink resin) was synthesized according to the Fmoc strategy on an Applied Biosystems 431A peptide synthesizer on a 0.25 mmol scale using the manufacturer-supplied “FastMoc UV” protocols which employ HBTU (2-(1H-Benzotriazol-1-yl-)-1,1,3,3 tetramethyluronium hexafluorophosphate) mediated couplings in NMP (N-methylpyrrolidone) and UV monitoring of the deprotection of the Fmoc protection group. The starting resin used for the synthesis was 0.50 g (4-((2′,4′-dimethoxyphenyl)-(Fmoc-amino)methyl)-phenoxypolystyrene resin (Rink resin) (Novabiochem) with a loading of 0.51 mmol/g. The protected amino acid derivatives used were Fmoc-Lys(Mtt)-OH, Fmoc-Trp(Boc)-OH, Fmoc-Arg(Pmc)-OH, Fmoc-D-Phe-OH, Fmoc-Hyp(tBu)-OH, Fmoc-Glu(2-phenylisopropyloxy)-OH and Fmoc-Nle-OH.

1.b The resin resulting from (1.a) was treated with 5×10 ml 2% trifluoroacetic acid (TFA), 2% triethylsilane (TES) in DCM during 60 minutes with regular mixing. The resin was washed with NMP, NMP with 5% DIEA and NMP. The peptide was cyclized using HOBt (1.0 mmol), (benzotriazol-1-yloxy)tripyrrolidinophosphonium hexafluorophosphate 1H-benzotriazol-1-yloxytripyrrolidinophosphonium hexafluorophosphate (PyBOP) (1.0 mmol) and DIEA (2.0 mmol) in NMP (5 ml) with regular mixing for 4 h. The resin was washed with NMP and DCM.

1.c The peptide was cleaved from the resin obtained from (1.c) by stirring for 60 minutes at room temperature with 10 ml of 2.5% water and 2.5% TES in TFA. The cleavage mixture was filtered and the filtrate was concentrated to approximately 1 ml by a stream of nitrogen. The crude peptide was precipitated from this oil with 50 ml diethyl ether and washed 3 times with 50 ml diethyl ether.

The crude cyclic peptide was purified by preparative RP-HPLC.

Synthesis of the Trimerisation Moiety

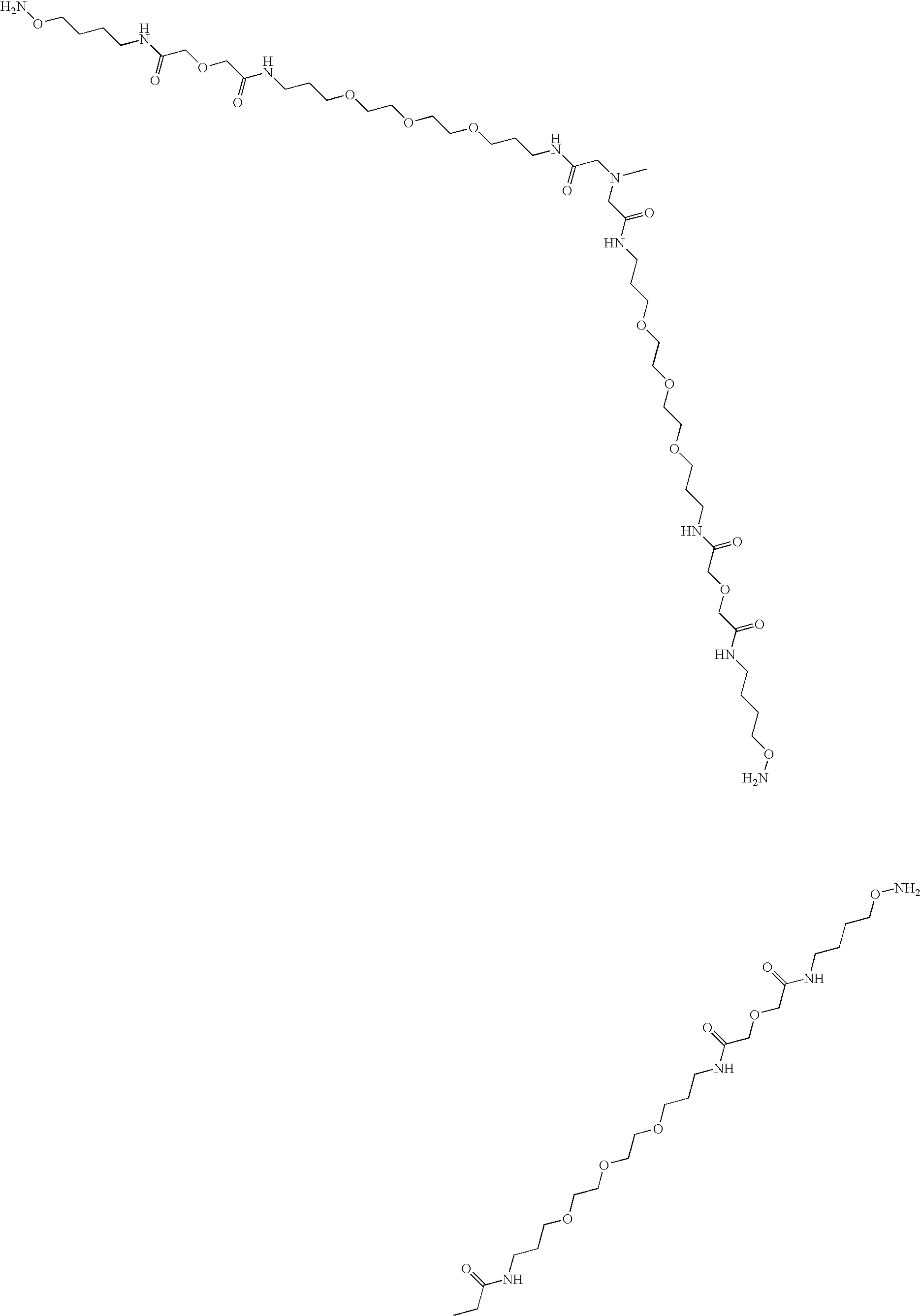

2.a Monoprotection of a Diamine.

50 g of the diamine (4,7,10-Trioxa-1,13-Tridecanediamine) was dissolved in 250 ml of dichlormethane. 17.5 mL of trifluoroacetic acid was added dropwise to the clear colorless solution. 49.5 g of di-tertbutyl-dicarbonate in 2000 ml of dichlormethane was added dropwise over 5 hours to the solution. The reaction mixture was stirred over night at room temperature and then concentrated in vacuum, yielding of clear yellow oil.

The oil was dissolved in dichlormethane and the desired compound was extracted into 2×0.1N HCl (250 ml).

The water phase was treated with 280 ml 1 N NaOH. The basic water phase was extraced twice with 250 ml of dichlormethane. The combined organic phases were washed with brine (200 ml). The dichloromethane was evaporated in vacuum yielding a clear light yellow oil.

2.b Attachment of the mono-boc-amine to the Central Unit.

1 g of nitrilotriacetic acid was dissolved in 15 mL of tetrahydrofuran. 4 g of 1-hydroxybenzotriazole (HOBt) and 2.6 mL of diisopropylcarbodiimide (DIC) were added and the mixture was stirred for 30 min. 5.4 g of the mono-boc-amine and 2.7 mL of diisopropylethylamine (DIPEA) were added to the active ester. The reaction mixture was stirred over night at room temperature.

The reaction mixture was concentrated in vacuum and apportioned between water and ethyl acetate. The ethyl acetate phase was concentrated in vacuo yeilding a semicrystalline oil in 10 g. The oil was cromatographed on Silica with 30% methanol in dichlormethane. The oil passed through the column as a plug. The fraction was concentrated in vacuum, and the remanence was redissolved in ethyl acetate. The solution was washed with sat. sodium hydrogen carbonate and finally with sat. sodium hydrogen sulphate. The ethyl acetate phase was concentrated in vacuo and then stripped with acetonitrile.

The remaining oil was dissolved in 100 mL of 50% trifluoroacetic acid in dichlormethane and then stirred at room temperature for 1 hour. The reaction mixture was concentrated in vacuo.

The mixture was apportioned between water and ethyl acetate. The water phase was washed twice with ethyl acetate and then lyophilised yielding a yellow oil in 9.5 g.

2.c Creation of the Anchoring Unit.

6 g of (4-aminobutoxy)carbamic acid 1,1-dimethylethyl ester (WO 2005014049 A2) was dissolved in 20 mL pyridine and 4.1 g diglycolic anhydride was added. The clear yellow solution was stirred at 100° C. for 3 hours. The reaction mixture was then poured into 0.5 N HCl (150 mL) and the water phase was extracted with dichloromethane. The organic phase was dried over magnesium sulfate and the solvent was removed in vacuum yielding a yellow oil.

2.d Attachment of the Anchoring Unit to the Trimerisation Moiety

0.66 g of the acid from 2.c was dissolved in 5 mL dichloromethane and 0.29 mL triethylamine was added. The solution was cooled on ice and 0.25 mL pivaloyl chloride in 5 mL dichlormethane was added. The mixture was stirred for 1 hour. 0.53 g of the tetramine from 2.b was dissolved in 5 mL together with 0.29 mL triethylamine. To this solution the mixed anhydride was added and the mixture was stirred over night.

The solvent was removed in vacuum and the yielding oil was purified by HPLC on reverse phase using acetonitrile/water+0.1% TFA. The boc-protecting groups were removed by treatment with 10% TFA in dichlormethane. The final product can be purified by reverse phase HPLC. Preferable the molecule is deprotected just before the trimerization and used unpurified.

Trimerization of the Peptide

3.a 21 mg of the peptide (1.c) was dissolved in 2 mL water and this solution was added to 6 mg of the unprotected trisoxiamine (2.d). The mixture was stirred for 1 hour at room temperature. The final product was then purified on reverse phase HPLC with acetonitril/water/0.1% TFA. MS-data: (M+4H)⁴⁺=1533.

### Pharmacological Methods

Assay (I)—Experimental Protocol for Efficacy Testing on Appetite with MC4 Analogues, Using an Ad Libitum Fed Rat Model.

TAC:SPRD rats or Wistar rats from M&B Breeding and Research Centre A/S, Denmark are used for the experiments. The rats have a body weight of 200-250 g at the start of the experiment. The rats arrive at least 10-14 days before start of the experiment with a body weight of 180-200 g. Each dose of compound is tested in a group of 8 rats. A vehicle group of 8 rats is included in each set of testing.

When the animals arrive they are housed individually in a reversed light/dark phase (lights off 7:30 am, lights on 7:30 pm), meaning that lights are off during daytime and on during nighttime. Since rats normally initiate food intake when light is removed, and eat the major part of their daily food intake during the night, this set-up results in an alteration of the initiation time for food intake to 7:30 am, when lights are switched off. During the acclimatization period of 10-14 days, the rats have free access to food and water. During this period the animals are handled at least 3 times. The experiment is conducted in the rats' home cages. Immediately before dosing, the rats are randomised to the various treatment groups (n=8) by body weight. They are dosed according to body weight at between 7:00 am and 7:45 am, with a 1-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually every hour for 7 hours, and then after 24 h and sometimes 48 h. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

Assay (II)—Melanocortin Receptor 3 and 5 (MC3 and MC5) cAMP Functional Assay using the AlphaScreen™ cAMP Detection Kit

The cAMP assays for MC3 and MC5 receptors are performed on cells (either HEK293 or BHK cells) stably expressing the MC3 and MC5 receptors, respectively. The receptors are cloned from cDNA by PCR and inserted into the pcDNA 3 expression vector. Stable clones are selected using 1 mg/ml G418.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and then resuspended in stimulation buffer to a final concentration of 1×10⁶ or 2×10⁶ cells/ml. 25 μl of cell suspension is added to the microtiter plates containing 25 μl of test compound or reference compound (all diluted in stimulation buffer). The plates are incubated for 30 minutes at room temperature (RT) on a plate-shaker set to a low rate of shaking. The reaction is stopped by adding 25 μl of acceptor beads with anti-cAMP, and 2 min later 50 μl of donor beads per well with biotinylated cAMP in a lysis buffer. The plates are then sealed with plastic, shaken for 30 minutes and allowed to stand overnight, after which they are counted in an Alpha™ microplate reader.

EC₅₀ values are calculated by non-linear regression analysis of dose/response curves (6 points minimum) using the Windows™ program GraphPad™ Prism (GraphPad™ Software, USA). All results are expressed in nM.

For measuring antagonistic activity in the MC3 functional cAMP assay, the MC3 receptors are stimulated with 3 nM α-MSH, and inhibited with increasing amounts of the potential antagonist. The IC₅₀ value for the antagonist is defined as the concentration that inhibits MC3 stimulation by 50%.

Assay (III)—Melanocortin Receptor 4 (MC4) cAMP Assay

BHK cells expressing the human MC4 receptor are stimulated with potential MC4 agonists, and the degree of stimulation of cAMP is measured using the Flash Plate® cAMP assay (NEN™ Life Science Products, cat. No. SMP004).

The human MC4 receptor-expressing BHK cells are produced by transfecting the cDNA encoding MC4 receptor into BHK570/KZ10-20-48, and selecting for stable clones expressing the MC4 receptor. The human MC4 receptor cDNA, as well as a CHO cell line expressing the MC4 receptor, may be purchased from Euroscreen™. The cells are grown in DMEM, 10% FCS, 1 mg/ml G418, 250 nM MTX and 1% penicillin/streptomycin.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and resuspended in stimulation buffer to a final concentration of 0.75×10⁶ cells/ml (consumption thereof: 7 ml per 96-well microtiter plate). 50 μl of cell suspension is added to the Flash Plate containing 50 μl of test compound or reference compound (all diluted in H₂O). The mixture is shaken for 5 minutes and then allowed to stand for 25 minutes at RT. The reaction is stopped by addition of 100 μl Detection Mix per well (Detection Mix=11 ml Detection Buffer+100 μl (˜2 μCi) cAMP [¹²⁵I] tracer). The plates are then sealed with plastic, shaken for 30 minutes, and allowed to stand overnight (or for 2 hours) and then counted in the Topcounter (2 min/well). In general, the assay procedure is as described in the Flash Plate kit-protocol (Flash Plate® cAMP assay (NEN™ Life Science Products, cat. No. SMP004)). However, the cAMP standards are diluted in 0.1% HSA and 0.005% Tween™ 20, and not in stimulation buffer.

EC₅₀ values are calculated by non-linear regression analysis of dose/response curves (6 points minimum) using the Windows™ program GraphPad™ Prism (GraphPad Software, USA). All results are expressed in nM.

### Assay (IV)—Melanocortin Receptor 1 (MC1) Binding Assay

The MC1 receptor binding assay is performed on BHK cell membranes stably expressing the MC1 receptor. The assay is performed in a total volume of 250 μl: 25 μl of ¹²⁵NDP-α-MSH (22 μM in final concentration), 25 μl of test compound/control and 200 μl of cell membrane (35 μg/ml). Test compounds are dissolved in DMSO. Radioactively labeled ligand, membranes and test compounds are diluted in buffer: 25 mM HEPES, pH 7.4, 0.1 mM CaCl₂, 1 mM MgSO₄, 1 mM EDTA, 0.1% HSA and 0.005% Tween™ 20. The samples are incubated at 30° C. for 90 min in Greiner microtiter plates, separated with GF/B filters that are pre-wetted for 60 min in 0.5% PEI, and washed 2-3 times with NaCl (0.9%) before separation of bound from unbound radiolabelled ligand by filtration. After filtration the filters are washed 10 times with ice-cold 0.9% NaCl. The filters are dried at 50° C. for 30 min, sealed, and 30 μl of Microscint 0 (Packard, cat. No. 6013616) is added to each well. The plates are counted in a Topcounter (1 min/well).

The data are analysed by non-linear regression analysis of binding curves, using the Windows™ program GraphPad™ Prism (GraphPad Software, USA).

### Assay (V)—Melanocortin Receptor 4 (MC4) Binding Assay

### In Vitro ¹²⁵NDP-α-MSH Binding to Recombinant BHK Cells Expressing Human MC4 Receptor (Filtration Assay).

The assay is performed in 5 ml minisorb vials (Sarstedt No. 55.526) or in 96-well filterplates (Millipore MADVN 6550), and using BHK cells expressing the human MC4 receptor (see Assay III, above). The BHK cells are kept at −80° C. until assay, and the assay is run directly on a dilution of this cell suspension, without further preparation. The suspension is diluted to give maximally 10% specific binding, i.e. to approx. 50-100 fold dilution. The assay is performed in a total volume of 200 μl:50 μl of cell suspension, 50 μl of ¹²⁵NDP-α-MSH (≈79 pM in final concentration), 50 μl of test compound and 50 μl binding buffer (pH 7) mixed and incubated for 2 h at 25° C. [binding buffer: 25 mM HEPES (pH 7.0), 1 mM CaCl₂, 1 mM MgSO₄, 1 mM EGTA, 0.02% Bacitracin and 0.2% BSA]. Test compounds are dissolved in H₂O or DMSO and diluted in binding buffer. Radiolabelled ligand and membranes are diluted in binding buffer. The incubation is stopped by dilution with 5 ml ice-cold 0.9% NaCl, followed by rapid filtration through Whatman GF/C filters pre-treated for 1 hour with 0.5% PEI. The filters are washed with 3×5 ml ice-cold NaCl. The radioactivity retained on the filters is counted using a Cobra II auto gamma counter.

The data are analysed by non-linear regression analysis of binding curves, using the Windows™ program GraphPad™ Prism (GraphPad Software, USA).

### Assay (VI)—Evaluation of Energy Expenditure

TAC:SPRD rats or Wistar rats from M&B Breeding and Research Centre A/S, Denmark are used. After at least one week of acclimatization, rats are placed individually in metabolic chambers (Oxymax system, Columbus Instruments, Columbus, Ohio, USA; systems calibrated daily). During the measurements, animals have free access to water, but no food is provided to the chambers. Light:dark cycle is 12 h:12 h, with lights being switched on at 6:00. After the animals have spent approx. 2 hours in the chambers (i.e. when the baseline energy expenditure is reached), test compound or vehicle are administered (po, ip or sc), and recording is continued in order to establish the action time of the test compound. Data for each animal (oxygen consumption, carbon dioxide production and flow rate) are collected every 10-18 min for a total of 22 hours (2 hours of adaptation (baseline) and 20 hours of measurement). Correction for changes in O₂ and CO₂ content in the inflowing air is made in each 10-18 min cycle.

Data are calculated per metabolic weight [(kg body weight)^{0.75}] for oxygen consumption and carbon dioxide production, and per animal for heat. Oxygen consumption (VO₂) is regarded as the major energy expenditure parameter of interest.

1. A compound of the general formula I 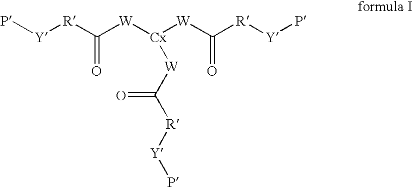
wherein Cx represents a center moiety having three independent arms;
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally, one or more groups B, said groups, when R′ contains one or more groups B, being linked in any order, with the proviso that that end of each R′ that is attached to the carbonyl group bound to W begins with a group A;
wherein A is 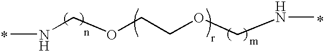
wherein r is 0 or an integer from 1 to 50,
and n and m independently are integers ≧2;
B is 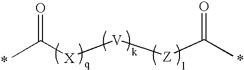
wherein X, V and Z independently are selected from —CR¹R²—, C₃₋₈-cycloalkylene,
C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl, C₂₋₆-alkenyl, C₃₋₈-cycloalkyl and C₄₋₈-cycloalkenyl, or R¹ and R² taken together form a C₂₋₆-alkylene bridge;
R″ is selected from hydrogen, C₁₋₆-alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and 1 are not all 0; and
Y′ is a moiety which is derived from a group Y, and to which a peptide moiety P′ derived from a peptide P that is a ligand for the MC4 receptor is covalently attached;
and pharmaceutically acceptable salts, prodrugs and solvates thereof.

2. A compound according to claim 1 which is represented by one of the following structures: 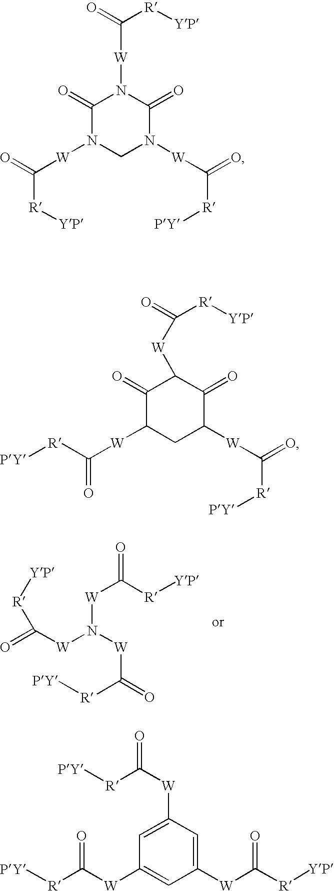
wherein R′, W, Y′ and P′ are as defined in claim 1.

3. A compound according to claim 1, wherein W is a bond or C₁₋₆-alkylene.

4. A compound according to claim 1 which is represented by one of the following structures: 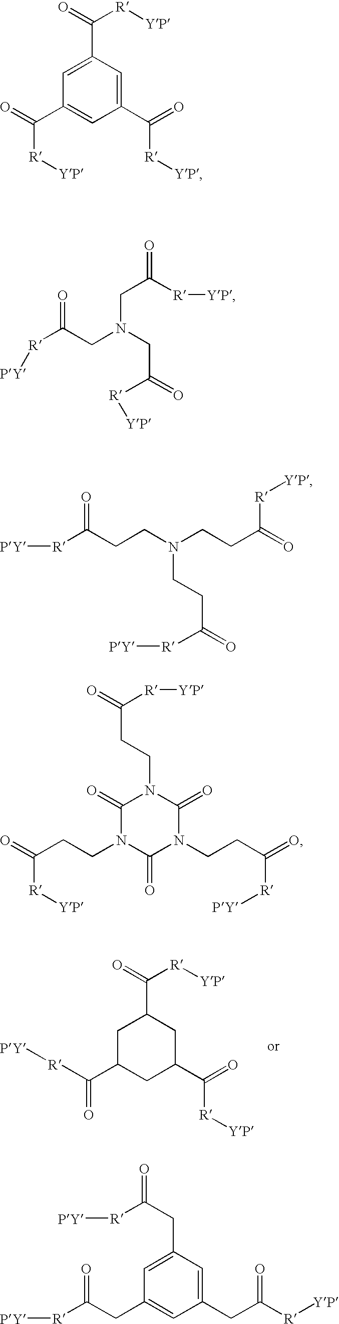
wherein R′, Y′ and P′ are as defined in claim 1.

5. A compound according to claim 1, wherein r is 0 or an integer from 1 to 25.

6. A compound according to claim 5, wherein r is 0 or an integer from 1 to 10.

7. A compound according to claim 5, wherein r is 0, 1, 2, 3, 4 or 5.

8. A compound according to claim 5, wherein r is 0, 1, 2 or 3.

9. A compound according to claim 1, wherein A is

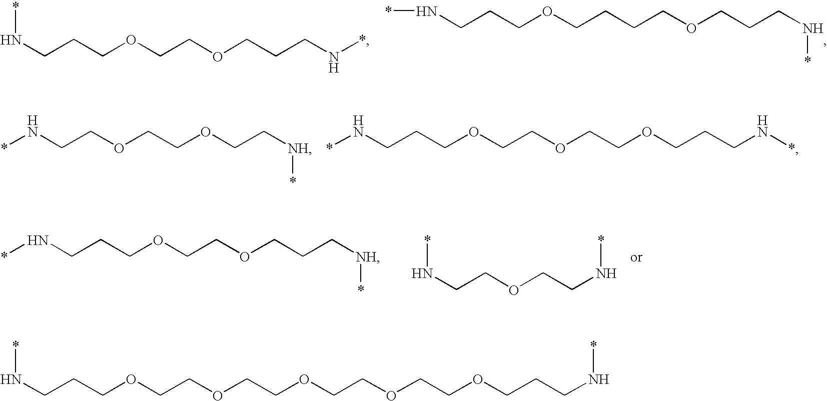

10. A compound according to claim 1, wherein X, V and Z independently represent —CR¹R²—, —O—, —S—, —NR″—, —OCH₂CH₂O—, —OCH₂CH₂—, —CH₂CH₂O—, —OCH₂— or
—CH₂O—, wherein R¹, R² and R″ are as defined in claim 1.

11. A compound according to claim 10, wherein X, V and Z independently represent —CR¹R²—.

12. A compound according to claim 10, wherein X, V and Z independently represent —OCH₂CH₂O—, —OCH₂— or —CH₂O—.

13. A compound according to claim 10, wherein X, V and Z taken together represent —(CH₂)₁₋₄—, —CH₂(OCH₂CH₂)₁₋₆OCH₂— or —CH₂OCH₂—.

14. A compound according to claim 13, wherein X, V and Z taken together represent —(CH₂)₃— or —CH₂OCH₂—.

15. A compound according to claim 10, wherein B is

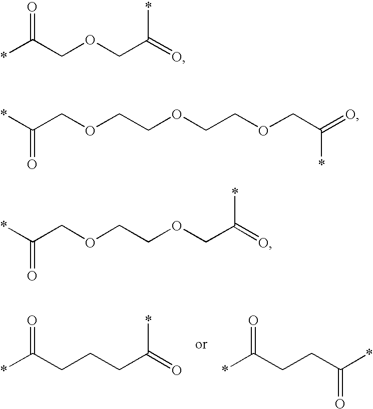

16. A compound according to claim 1, wherein X, V and Z independently represent arylene or heteroarylene.

17. A compound according to claim 16, wherein V is a phenylene.

18. A compound according to claim 17, wherein V is o-phenylene, m-phenylene or p-phenylene.

19. A compound according to claim 1, wherein V is C₃₋₈-cycloalkylene or C₄₋₈-cycloalkenylene.

20. A compound according to claim 19, wherein V is a cyclopentylene, a cyclohexylene or a cyclohexenylene.

21. A compound according to claim 1, wherein V is a di-radical derived from morpholine, piperazine, dioxan or thiophene.

22. A compound according to claim 1, wherein each Y independently is 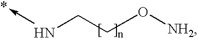
wherein n is an integer ≧0.

23. A compound according to claim 22, wherein all three Y groups are

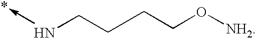

24. A compound according to claim 1, wherein each group R′ independently is selected from the following: -A-, -A-B-, -A-B-A-, -A-B-A-B-, -A-B-A-B-A- and -A-B-A-B-A-B-.

25. A compound according to claim 1, wherein all three R′ groups are identical.

26. A compound according to claim 1, wherein two of the three R′ groups are identical.

27. A compound according to claim 1, containing three different R′ groups.

28. A compound according to claim 1, wherein each P independently is
R¹-X-X¹-X²-X³-X⁴-X⁵-X⁶-X⁷-X⁸-X⁹-X¹⁰-X¹¹-R² (formula A)
wherein R¹, which is bonded to an N-terminal NH₂-group, is either absent or represents C₁₋₄-alkanoyl or an anchoring group R^{X}, said anchoring group R^{X} optionally being attached to X via a linker S, said linker S, if present, being selected from β-alanine, Glu, Gly-Gln, Gly-Glu, Gly-His and 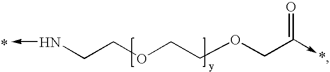
wherein y is 1, 2, 3, 4 or 5;
X represents a bond, an amino acid residue, or a di- or tri-peptide residue, wherein the amino acid(s) may be naturally occurring or synthetic;
X¹ represents a bond or an amino acid residue with a functional group in the side chain to which an anchoring group Rx may be attached, optionally via a linker S, said linker S, if present, being selected from β-alanine, Glu, Gly-Gln, Gly-Glu, Gly-His and 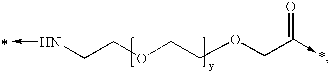
wherein y is 1, 2, 3, 4 or 5;
and wherein said anchoring group R^{x} represents a group of formula X, Xa or Xb 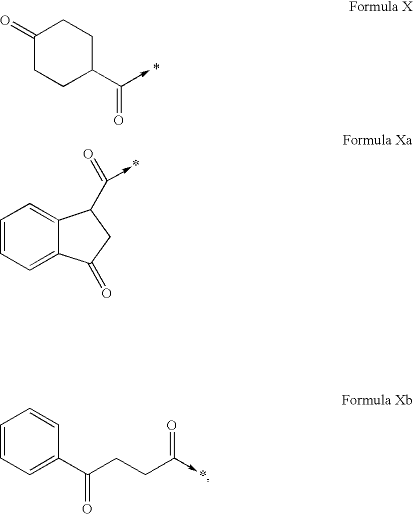
or R^{x} represents a C₂₋₆-alkanoyl or C₃₋₆-alkenoyl bearing a keto or aldehyde function which may react with said group Y to form Y′P′;
X² represents a bond, an amino acid residue, or a di-, tri- or tetra-peptide residue, wherein the amino acid(s) may be naturally occurring or synthetic;
X³ represents a bond or an amino acid residue, said amino acid residue optionally being capable of forming a bridge to X¹⁰;
X⁴ represents a bond, an amino acid residue or a di-peptide residue, wherein the amino acid(s) may be naturally occurring or synthetic;
X⁵ represents an amino acid residue selected from His, Ala, Nle, Met, Met(O), Met(O₂), Gln, Gln(F-alkyl), Gln(F-aryl), Asn, Asn(ε-alkyl), Asn(ε-aryl), Ser. Thr, Cys, F-Pro, Pro, Hyp,
(S)-1,2,3,4-tetrahydroisoquinoline-3-carboxylic acid, Trp, 1-naphthylalanine, 2-naphthylalanine, 2-pyridylalanine, 3-pyridylalanine, 4-pyridylalanine, 2-thienylalanine, 3-thienylalanine, 4-thiazolylalanine, 2-furylalanine, 3-furylalanine and Phe, wherein the phenyl moiety of said Phe is optionally substituted with halogen, hydroxyl, alkoxy, nitro, benzoyl, methyl, trifluoromethyl or cyano;
X⁶ represents D-Phe, wherein the phenyl moiety of said D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, an amino acid residue or a di-peptide residue, wherein the amino acid(s) may be naturally occurring or synthetic;
X¹⁰ represents a bond or an amino acid residue, said amino acid residue optionally being capable of forming a bridge to X³;
X¹¹ represents a bond, an amino acid residue or a di-peptide residue, wherein the amino acid(s) may be naturally occurring or synthetic;
R² represents —OH or —NR⁵R⁶, wherein R⁵ and R⁶ independently represent hydrogen, C₁₋₈alkyl, C₂₋₈alkenyl or C₂₋₈alkynyl;
wherein each P independently is optionally cyclized from X³ to X¹⁰ via a lactam or a disulfide bridge;
with the proviso that each P comprises only one anchoring group;
and with the further proviso that each P comprises at least 7 amino acid residues.

29. A compound according to claim 28, wherein all three P are identical.

30. A compound according to claim 28, wherein there is a bridge between X³ and X¹⁰ rendering the compound of formula I cyclic, either by the presence of a disulfide bridge formed between X³ and X¹⁰ moieties independently selected from Cys and homoCys, or by the presence of a lactam bond formed between a carboxylic acid moiety in the side-chain of X³ and an amine moiety in the side-chain of X¹⁰, or between a carboxylic acid moiety in the side-chain of X¹⁰ and an amine moiety in the side-chain of X³.

31. A compound according to claim 28, wherein X² represents Nle.

32. A compound according to claim 28, wherein X³ represents Glu or Asp, and X¹⁰ represents Lys, Orn, Dab or Dap.

33. A compound according to claim 32, wherein X³ represents Glu or Asp, and X¹⁰ represents Lys.

34. A compound according to claim 32, wherein X³ represents Glu, and X¹⁰ represents Lys.

35. A compound according to claim 28, wherein R² represents —NH₂.

36. A compound according to claim 28, wherein X⁶-X⁷-X⁸-X⁹-X¹⁰ represents D-Phe-Arg-Trp-Lys.

37. A method of delaying the progression from IGT to type 2 diabetes, comprising administering to a patient in need thereof an effective amount of a compound of the general formula I 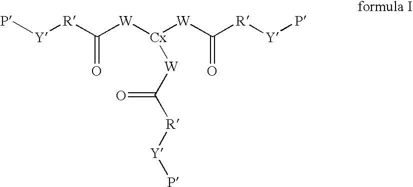
wherein Cx represents a center moiety having three independent arms,
each W independently represents C₁₋₆-alkylene, C₁₋₆-alkyleneoxy, C₂₋₆-alkenylene, C₂₋₆-alkylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups when R′ contains one or more groups B, being linked in any order with the proviso that that end of each R′ that is attached to the carbonyl group bound to W begins with a group A;
wherein A is 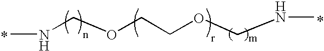
wherein r is 0 or an integer from 1 to 50,
and n and m independently are integers ≧2;
B is 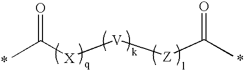
wherein X, V and Z independently are selected from —CR¹CR²-cycloalkylene C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆alkyl C₂₋₆-alkenyl, C₃₋₈-cycloalkyl and C₄₋₈cycloalkenyl or R¹ and R² taken together form a C₂₋₆-alkylene bridge;
R″ is selected from hydrogen, C₁₋₆alkyl C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts, prodrugs and solvates thereof, optionally in combination with one or more additional therapeutically active compounds.

38. A method of delaying the progression from type 2 diabetes to insulin-requiring diabetes, comprising administering to a patient in need thereof an effective amount of a compound of the general formula I 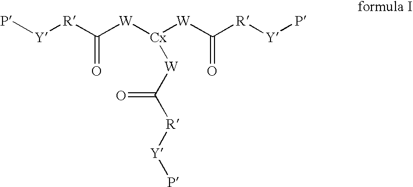
wherein Cx represents a center moiety having three independent arms,
each W independently represents C₁₋₆-alkylene, C₁₋₆-alkyleneoxy, C₂₋₆-alkenylene, C₂₋₆-alkylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally, one or more groups B, said groups, when R′ contains one or more groups B, being linked in any order, with the proviso that that end of each R′ that is attached to the carbonyl group bound to W begins with a group A;
wherein A is 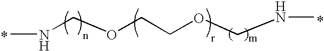
wherein r is 0 or an integer from 1 to 50,
and n and m independently are integers ≧2;
B is 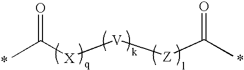
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆alkyl, C₂₋₆-alkenyl, C₃₋₈-cycloalkyl and C₄₋₈cycloalkenyl, or R¹ and R² taken together form a C₂₋₆-alkylene bridge;
R″ is selected from hydrogen, C₁₋₆alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts, prodrugs and solvates thereof, optionally in combination with one or more additional therapeutically active compounds.

39. A method of treating obesity or preventing overweight, comprising administering to a patient in need thereof an effective amount of a compound of the general formula I 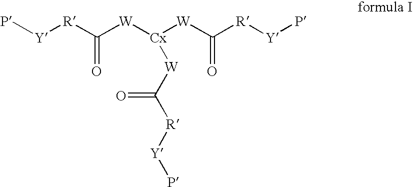
wherein Cx represents a center moiety having three independent arms;
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups when R′ contains one or more groups B, being linked in any order with the proviso that that end of each R′ that is attached to the carbonyl group bound to W begins with a group A;
wherein A is 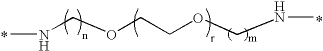
wherein r is 0 or an integer from 1 to 50,
and n and m independently are integers ≧2;
B is 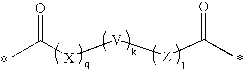
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl C₂₋₆-alkenyl, C₃₋₈-cycloalkyl and C₄₋₈cycloalkenyl or R¹ and R² taken together form a C₂₋₆-alkylene bridge;
R″ is selected from hydrogen, C₁₋₆alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof, optionally in combination with one or more additional therapeutically active compounds.

40. A method of regulating appetite, comprising administering to a patient in need thereof an effective amount of a compound of the general formula I 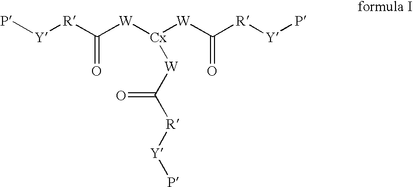
wherein Cx represents a center moiety having three independent arms;
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally, one or more groups B, said groups, when R′ contains one or more groups B, being linked in any order, with the proviso that that end of each R′ that is attached to the carbonyl group bound to W begins with a group A;
wherein A is 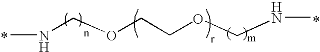
wherein r is 0 or an integer from 1 to 50,
and n and m independently are integers ≧2;
B is 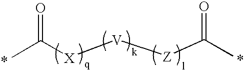
wherein X, V and Z independently are selected from —CR¹R²—, C₃₋₈-cycloalkylene C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl C₂₋₆-alkenyl, C₃₋₈-cycloalkyl and C₄₋₈-cycloalkenyl, or R¹ and R² taken together form a C₂₋₆-alkylene bridge;
R″ is selected from hydrogen, C₁₋₆alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof, optionally in combination with one or more additional therapeutically active compounds.

41. A method of inducing satiety, comprising administering to a patient in need thereof an effective amount of a compound of the general formula I 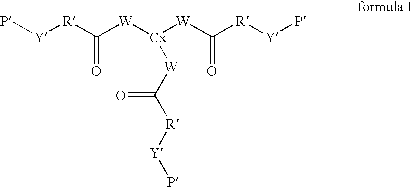
wherein Cx represents a center moiety having three independent arms;
each W independently represents C₁₋₆-alkylene, C₁₋₆-alkyleneoxy, C₂₋₆-alkenylene, C₂₋₆-alkylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups when R′ contains one or more groups B, being linked in any order, with the proviso that that end of each R′ that is attached to the carbonyl group bound to W begins with a group A;
wherein A is 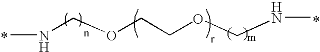
wherein r is 0 or an integer from 1 to 50,
and n and m independently are integers ≧2;
B is 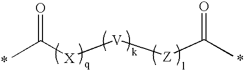
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl C₂₋₆-alkenyl, C₃₋₈-cycloalkyl and C₄₋₈cycloalkenyl, or R¹ and R² taken together form a C₂₋₆-alkylene bridge;
R″ is selected from hydrogen, C₁₋₆alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts, prodrugs and solvates thereof, optionally in combination with one or more additional therapeutically active compounds.

42. A method of preventing weight gain after successfully having lost weight, comprising administering to a patient in need thereof an effective amount of a compound of the general formula I 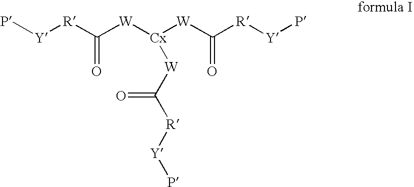
wherein Cx represents a center moiety having three independent arms,
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups, when R′ contains one or more groups B, being linked in any order, with the proviso that that end of each R′ that is attached to the carbonyl group bound to W begins with a group A;
wherein A is 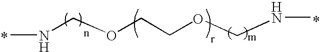
wherein r is 0 or an integer from 1 to 50,
and n and m independently are integers ≧2;
B is 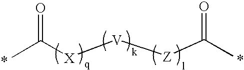
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl C₂₋₆-alkenyl, C₃₋₈-cycloalkyl and C₄₋₈cycloalkenyl, or R¹ and R² taken together form a C₂₋₆-alkylene bridge;
R″ is selected from hydrogen, C₁₋₆-alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts, prodrugs and solvates thereof, optionally in combination with one or more additional therapeutically active compounds.

43. A method of increasing energy expenditure, comprising administering to a patient in need thereof an effective amount of a compound of the general formula I 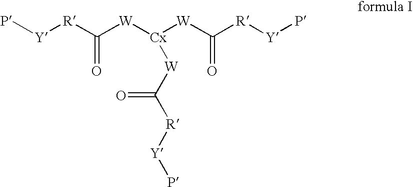
wherein Cx represents a center moiety having three independent arms;
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally, one or more groups B, said groups, when R′ contains one or more groups B, being linked in any order, with the proviso that that end of each R′ that is attached to the carbonyl group bound to W begins with a group A;
wherein A is 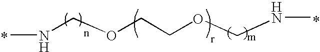
wherein r is 0 or an integer from 1 to 50,
and n and m independently are integers ≧2;
B is 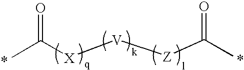
wherein X, V and Z independently are selected from —CR¹R²—, C₃₋₈-cycloalkylene C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl, C₂₋₆-alkenyl, C₃₋₈-cycloalkyl and C₄₋₈-cycloalkenyl, or R¹ and R² taken together form a C₂₋₆-alkylene bridge;
R″ is selected from hydrogen, C₁₋₆-alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that g, k and l are not all 0; and
Y′ is a moiety which is derived from a group Y, and to which a peptide moiety P′ derived from a peptide P that is a ligand for the MC4 receptor is covalently attached;
and pharmaceutically acceptable salts, prodrugs and solvates thereof, optionally in combination with one or more additional therapeutically active compounds.

44. A method of treating a disease or state related to overweight or obesity, comprising administering to a patient in need thereof an effective amount of a compound of the general formula I 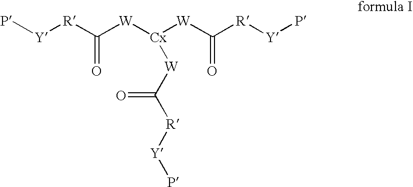
wherein Cx represents a center moiety having three independent arms;
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups, when R′ contains one or more groups B, being linked in any order, with the proviso that that end of each R′ that is attached to the carbonyl group bound to W begins with a group A;
wherein A is 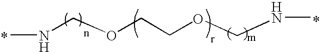
wherein r is 0 or an integer from 1 to 50,
and n and m independently are integers ≧2;
B is 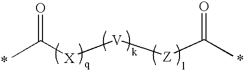
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl, C₂₋₆-alkenyl, C₃₋₈-cycloalkyl and C₄₋₈cycloalkenyl or R¹ and R² taken together form a C₂₋₆-alkylene bridge;
R″ is selected from hydrogen, C₁₋₆-alkyl C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof, optionally in combination with one or more additional therapeutically active compounds.

45. A method of treating bulimia, comprising administering to a patient in need thereof an effective amount of a compound of the general formula I 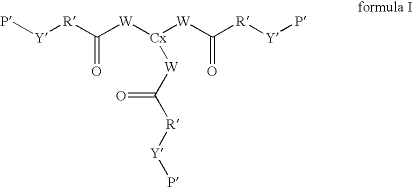
wherein Cx represents a center moiety having three independent arms,
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups when R′ contains one or more groups B, being linked in any order with the proviso that that end of each R′ that is attached to the carbonyl group bound to W begins with a group A;
wherein A is 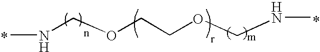
wherein r is 0 or an integer from 1 to 50,
and n and m independently are integers ≧2;
B is 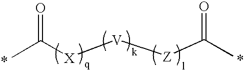
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl, C₂₋₆-alkenyl, C₃₋₈-cycloalkyl and C₄₋₈cycloalkenyl, or R¹ and R² taken together form a C₂₋₆-alkylene bridge;
R″ is selected from hydrogen, C₁₋₆-alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof, optionally in combination with one or more additional therapeutically active compounds.

46. A method of treating binge-eating, comprising administering to a patient in need thereof an effective amount of a compound of the general formula I 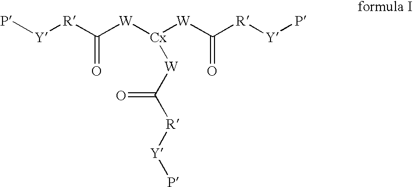
wherein Cx represents a center moiety having three independent arms,
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups when R′ contains one or more groups B, being linked in any order, with the proviso that that end of each R′ that is attached to the carbonyl group bound to W begins with a group A;
wherein A is 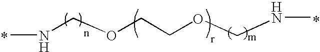
wherein r is 0 or an integer from 1 to 50,
and n and m independently are integers ≧2;
B is 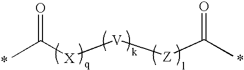
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl C₂₋₆-alkenyl, C₃₋₈-cycloalkyl and C₄₋₈-cycloalkenyl, or R¹ and R² taken together form a C₂₋₆-alkylene bridge;
R″ is selected from hydrogen, C₁₋₆alkyl C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof, optionally in combination with one or more additional therapeutically active compounds.

47. A method of treating a disease or state selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and risk of premature death, comprising administering to a patient in need thereof an effective amount of a compound of the general formula I 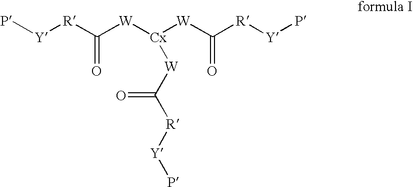
wherein Cx represents a center moiety having three independent arms,
each W independently represents C₁₋₆-alkylene, C₁₋₆-alkyleneoxy, C₂₋₆-alkenylene C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally one or more groups B, said groups when R′ contains one or more groups B, being linked in any order with the proviso that that end of each R′ that is attached to the carbonyl group bound to W begins with a group A;
wherein A is 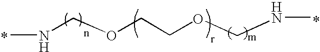
wherein r is 0 or an integer from 1 to 50,
and n and m independently are integers ≧2;
B is 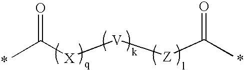
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl, C₂₋₆-alkenyl, C₃₋₈-cycloalkyl and C₄₋₈-cycloalkenyl, or R¹ and R² taken together form a C₂₋₆-alkylene bridge;
R″ is selected from hydrogen, C₁₋₆-alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof, optionally in combination with one or more additional therapeutically active compounds.

48. A method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death, comprising administering to an obese patient in need thereof an effective amount of a compound of the general formula I 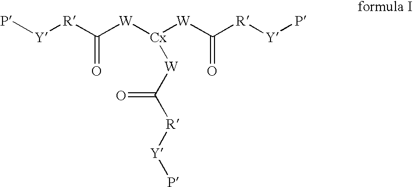
wherein Cx represents a center moiety having three independent arms,
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally, one or more groups B, said groups when R′ contains one or more groups B, being linked in any order, with the proviso that that end of each R′ that is attached to the carbonyl group bound to W begins with a group A;
wherein A is 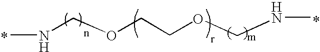
wherein r is 0 or an integer from 1 to 50,
and n and m independently are integers ≧2;
B is 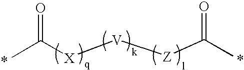
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen C₁₋₆-alkyl, C₂₋₆-alkenyl, C₃₋₈-cycloalkyl and C₄₋₈cycloalkenyl, or R¹ and R² taken together form a C₂₋₆-alkylene bridge;
R″ is selected from hydrogen, C₁₋₆alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof, optionally in combination with one or more additional therapeutically active compounds.

49. A method according to claim 37, wherein said additional therapeutically active compound is selected from antidiabetic agents, antihyperlipidemic agents, antiobesity agents, antihypertensive agents and agents for the treatment of complications resulting from, or associated with, diabetes.

50. A method of activating MC4 in a subject, comprising administering to said subject an effective amount of a compound of the general formula I 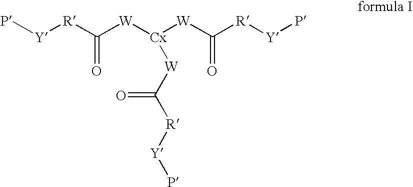
wherein Cx represents a center moiety having three independent arms;
each W independently represents C₁₋₆-alkylene, C₁₋₆-alkyleneoxy, C₂₋₆-alkenylene, C₂₋₆-alkynylene, hydroxy-C₁₋₆-alkylene, hydroxy-C₂₋₆-alkenylene, C₂₋₆-alkenyleneoxy, C₂₋₆-alkanoylene, C₂₋₆-alkenoylene or a bond,
each R′ independently consists of a chain of one or more groups A and, optionally, one or more groups B, said groups, when R′ contains one or more groups B, being linked in any order, with the proviso that that end of each R′ that is attached to the carbonyl group bound to W begins with a group A;
wherein A is 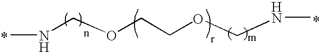
wherein r is 0 or an integer from 1 to 50,
and n and m independently are integers ≧2;
B is 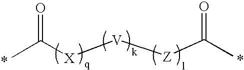
wherein X, V and Z independently are selected from —CR¹R²—, C₃₋₈-cycloalkylene, C₄₋₈-cycloalkenylene, arylene, heteroarylene, heterocyclylene, —O—, —S—, —N(R″)—, —OCH₂CH₂O—, —OCH₂— and —CH₂O—;
R¹ and R² independently are selected from hydrogen, C₁₋₆-alkyl, C₂₋₆-alkenyl, C₃₋₈-cycloalkyl and C₄₋₈cycloalkenyl, or R¹ and R² taken together form a C₂₋₆-alkylene bridge;
R″ is selected from hydrogen, C₁₋₆alkyl, C₂₋₆-alkenyl, C₃₋₈-cycloalkyl and C₄₋₈-cycloalkenyl;
q, k and l independently are selected from 0, 1, 2, 3, 4, 5 and 6, with the proviso that q, k and l are not all 0; and
Y′ is a moiety which is derived from a group Y, and to which a peptide moiety P′ derived from a peptide P that is a ligand for the MC4 receptor is covalently attached;
and pharmaceutically acceptable salts prodrugs and solvates thereof.

51. A method according to claim 37, wherein said compound is administered to said patient in a unit dosage form comprising about 0.05 mg to about 1000 mg of said compound.

52. A method according to claim 37, wherein said compound is administered by parenteral or sublingual administration.

53. (canceled)

54. (canceled)

55. (canceled)
